(19) United States
(12) Patent Application Publication
(10) Pub. No.: US 2026/0167957 A1
(43) Pub. Date: Jun. 18, 2026

(54) METHOD AND REAGENT FOR PREPARING SEQUENCING LIBRARY

(71) Applicant: MGI TECH CO., LTD., Shenzhen (CN)

(72) Inventors: Xia ZHAO, Shenzhen (CN); Qianyu SHI, Shenzhen (CN); Meihua GONG, Shenzhen (CN); Yinling LUO, Shenzhen (CN); Ziling LI, Shenzhen (CN); Qiaoling LI, Shenzhen (CN); Yicong WANG, Shenzhen (CN); Hong ZHANG, Shenzhen (CN); Yinqing WU, Shenzhen (CN)

(73) Assignee: MGI TECH CO., LTD., Shenzhen (CN)

(21) Appl. No.: 19/533,720

(22) Filed: Feb. 9, 2026

Related U.S. Application Data
(63) Continuation of application No. PCT/CN2023/134793, filed on Nov. 28, 2023.

(51) Int. Cl. C12N 15/10

(52) U.S. Cl. CPC C12N 15/1093

(57) ABSTRACT

The present disclosure relates to the technical field of sequencing, and in particular to a method and reagent for preparing a sequencing library. Provided in the present disclosure is a method for preparing a sequencing library. According to the method, by means of further adding an additive or an additive combination capable of reducing the DNA secondary structure and/or reducing the degradation of single-stranded DNA in the single-chain circularization reaction and/or the rolling circle amplification reaction, and further adding an additive or an additive combination capable of reducing the DNA secondary structure and reducing the degradation of single-stranded DNA and/or reducing the stability of the double helix structure of double-stranded DNA in the rolling circle amplification reaction of the double-stranded circular library, the ability of the DNA ligase and/or the strand displacement DNA polymerase to bind to a target nucleic acid is enhanced.

### CROSS-REFERENCE TO RELATED APPLICATIONS

This application is a continuation of International Patent Application No. PCT/CN2023/134793 filed on Nov. 28, 2023, the entire contents of which are incorporated herein by reference.

### TECHNICAL FIELD

The present disclosure relates to the technical field of sequencing, and in particular to a method and reagent for preparing a sequencing library.

### BACKGROUND

Methods for sequencing based on a circular library, either through library amplification or through direct sequencing without amplification, require circularization of the linear DNA to which sequencing anchor adapters have been added. The circularization process includes two types: (1) converting denatured single-stranded linear DNA into single-stranded circular DNA; and (2) converting double-stranded linear DNA into double-stranded circular DNA.

In the case that a circular library is used as the template for library amplification sequencing, a rolling circle amplification (RCA) reaction is required. In contrast, in the case that the circular library is directly sequenced, no RCA reaction is needed and single-molecule sequencing is performed directly.

In general, the circularization process is affected by multiple factors, including the length of linear DNA, the secondary structures of base sequences with different combinations and arrangements, the pH environment of substrates and enzyme in the enzymatic circularization reaction, the temperature, the intrinsic activity of enzyme, and the working concentration of substrates. As a result, the efficiency of converting linear DNA into circular DNA varies.

Current circularization methods have relatively low circularization efficiency. For example, in PCR-based library construction, the circularization efficiency of single-stranded DNA is approximately 10-20%, and that of double-stranded DNA is approximately 20-40%. Theoretically, if all single-stranded molecules are circularized, the theoretical maximum circularization efficiency can reach 50%; if all double-stranded molecules are circularized, the theoretical maximum circularization efficiency can reach 100%.

In the case that a circular library is used as the template for library amplification sequencing, the rolling circle replication of circular DNA is also affected by multiple factors, including the secondary structure of the template, the pH environment of substrates and enzyme in the RCA enzymatic reaction, the temperature, the intrinsic activity of enzyme, and the working concentration of substrates. As a result, the primer-template binding efficiency and the efficiency and fidelity of the RCA reaction vary. The subsequent sequencing processes are continually affected by multiple factors including the secondary structure of the template, the pH environment of substrates and enzyme in the sequencing enzymatic reaction, the temperature, the intrinsic activity of enzyme, and the working concentration of substrates, resulting in variations in the efficiency and stability of primer-template binding, as well as the efficiency and fidelity of the sequencing enzymatic reaction.

Although, to overcome the above difficulties, circular library sequencing employs optimized formulations which address a series of issues caused by these influencing factors to a large extent, the low circularization efficiency and poor sequencing quality in special sequence regions (e.g., stem-loop structures, consecutive base sequences, or palindromic sequences) remain challenges that require to be optimized and solved in current circular library sequencing. In particular, for methylation libraries, most cytosines (C) in the genome are converted to thymine (T) (e.g., the GC content of human genomic DNA is approximately 42%, with a C content of approximately 21%; if 5% of these Cs are methylated, then after unmethylated Cs are converted to uracil (U), the methylation library insert DNA contains approximately 1% C and approximately 49% T). This results in low signal intensity in the C channel and poor sequencing quality. In addition, single-stranded circular libraries (especially methylation libraries, where most of the Cs are converted to Ts and secondary structures of the sequence are reduced, leaving most regions in a loose conformation) are susceptible to degradation during reactions due to their relaxed single-stranded conformations, which adversely affects library quality.

Therefore, there is an urgent need for a method for improving library circularization efficiency and sequencing quality, especially for methylation libraries.

### SUMMARY

The present disclosure aims to solve at least one of the technical problems in the related art to some extent.

To this end, provided in a first aspect of the present disclosure is a method for preparing DNA nanoballs (DNB), the method including:
(a) performing a circularization reaction on a linear DNA library to obtain a circular DNA library, wherein the circularization reaction includes a single-stranded circularization reaction or a double-stranded circularization reaction, and the circular DNA library comprises a single-stranded circular DNA library or a double-stranded circular DNA library; and
(b) performing a rolling circle amplification reaction on the circular DNA library to obtain DNB.

In the case that the circularization reaction in step (a) is a single-stranded circularization reaction,
step (a) further comprises adding at least one of the following additives to a single-stranded circularization reaction to obtain a single-stranded circular DNA library with higher yield and more uniform coverage:
1) an additive capable of reducing DNA secondary structure; or
2) an additive capable of reducing DNA secondary structure and maintaining stability of single-stranded DNA, and/or
step (b) further comprises performing a rolling circle amplification reaction using the single-stranded circular DNA library from step (a), wherein at least one of the following additives is added in the rolling circle amplification reaction:
3) an additive capable of reducing DNA secondary structure; or
4) an additive capable of reducing DNA secondary structure and maintaining stability of single-stranded DNA.

In the case that the circularization reaction in step (a) is a double-stranded circularization reaction,
step (b) further comprises performing a rolling circle amplification reaction using the double-stranded circular DNA library from step (a), wherein at least one of the following additives is added in the rolling circle amplification reaction:
5) an additive capable of reducing DNA secondary structure and maintaining stability of single-stranded DNA; or
6) an additive capable of reducing stability of the double helix structure of circular double-stranded DNA.

Provided in the present disclosure is a method for improving sequencing quality. The method increases the binding ability of DNA ligase and/or strand-displacing DNA polymerase to target nucleic acids by adding an additive capable of reducing DNA secondary structure during the circularization reaction and/or rolling circle amplification (RCA). These additives facilitate the processing of DNA sequence fragments that are difficult to unwind and/or prone to forming secondary structures, such as those having high GC content, continuous AT or GC repeat regions, or palindromic sequences. In addition, by introducing an additive that stabilizes single-stranded structures and prevents their degradation, the method enhances the stability of DNA sequence fragments having low GC content and relatively loose structural conformations thereby reducing the likelihood of degradation. Further, by introducing an additive that reduces the stability of double-stranded DNA helices, the method improves strand separation efficiency and amplification uniformity of double-stranded circular libraries during RCA or other isothermal amplification processes. As a result, the method effectively enhances the circularization efficiency and enrichment uniformity of library regions with different GC contents and distinct sequence features, thereby improving sequencing quality, sequencing data yield, and sequencing data coverage uniformity. In particular, when applied to methylation sequencing libraries, the method—combined with an optimized base-calling algorithm—can significantly improve sequencing yield, sequencing data quality, and sequencing data coverage uniformity.

Provided in a second aspect of the present disclosure is a method for rolling circle amplification, comprising performing a rolling circle amplification reaction on a DNA library to obtain single-stranded linear DNA.

In the case that the DNA library is a single-stranded circular DNA library, at least one of the following additives is added in the rolling circle amplification reaction:
7) an additive capable of reducing DNA secondary structure; or
8) an additive capable of reducing DNA secondary structure and maintaining stability of single-stranded DNA.

In the case that the DNA library is a double-stranded circular DNA library, at least one of the following additives is added in the rolling circle amplification reaction:
9) an additive capable of reducing DNA secondary structure and maintaining stability of single-stranded DNA; or
10) an additive capable of reducing stability of the double helix structure of circular double-stranded DNA.

According to an embodiment of the present disclosure, the linear DNA library comprises not only whole-genome methylation libraries and targeted methylation libraries, but also any of the following: whole-genome WGS libraries prepared by PCR and/or PCR-free methods, targeted capture libraries (including exome libraries and/or panel libraries), amplicon libraries, RNA libraries, and stLFR libraries.

According to an embodiment of the present disclosure, the additive capable of reducing DNA secondary structure comprises at least one of a non-ionic detergent, betaine, or dimethyl sulfoxide (DMSO).

According to an embodiment of the present disclosure, the non-ionic detergent comprises at least one of Triton-X100, Tween-20, or NP40.

According to an embodiment of the present disclosure, the additive capable of reducing DNA secondary structure and maintaining stability of single-stranded DNA comprises at least one of single-stranded DNA binding protein (SSB) and heat shock protein (HSP).

According to an embodiment of the present disclosure, the additive capable of reducing stability of the double helix structure of circular double-stranded DNA comprises at least one of formamide, ammonium sulfate, urea, or tetramethylammonium hydroxide (TMAH).

According to an embodiment of the present disclosure, the additive in the single-stranded circularization reaction comprises at least one of SSB, a non-ionic detergent, betaine, or DMSO.

According to an embodiment of the present disclosure, the additive in the single-stranded circularization reaction is a mixture of a non-ionic detergent and betaine.

According to an embodiment of the present disclosure, the additive in the single-stranded circularization reaction is a mixture of a non-ionic detergent and DMSO.

According to an embodiment of the present disclosure, the additive in the single-stranded circularization reaction is a mixture of SSB and a non-ionic detergent and/or betaine.

According to an embodiment of the present disclosure, the additive in the rolling circle amplification reaction for the single-stranded circular DNA library comprises at least one of SSB, or a mixture of SSB and a non-ionic detergent and/or betaine.

According to an embodiment of the present disclosure, the additive in the rolling circle amplification reaction for the double-stranded circular DNA library comprises at least one of SSB or formamide.

According to an embodiment of the present disclosure, the working concentration of SSB added in the single-stranded circularization reaction is 5 ng/μL to 50 ng/μL.

According to an embodiment of the present disclosure, the working concentration of the non-ionic detergent added in the single-stranded circularization reaction is 0.05 vol % to 2.00 vol %.

According to an embodiment of the present disclosure, the working concentration of DMSO added in the single-stranded circularization reaction is 1.5 vol % to 4.0 vol %.

According to an embodiment of the present disclosure, the working concentration of betaine added in the single-stranded circularization reaction is 0.1 M to 1 M.

According to an embodiment of the present disclosure, the working concentration of SSB added in the rolling circle amplification reaction for the single-stranded circular DNA library is 5 ng/μL to 130 ng/μL.

According to an embodiment of the present disclosure, the working concentration of betaine added in the rolling circle amplification reaction for the single-stranded circular DNA library is 0.1 M to 1 M.

According to an embodiment of the present disclosure, the working concentration of the non-ionic detergent added in the rolling circle amplification reaction for the single-stranded circular DNA library is 0.05 vol % to 2.00 vol %.

According to an embodiment of the present disclosure, the working concentration of SSB added in the rolling circle amplification reaction for the double-stranded circular DNA library is 5 ng/μL to 130 ng/μL.

According to an embodiment of the present disclosure, the working concentration of formamide added in the rolling circle amplification reaction for the double-stranded circular DNA library is 1.5 vol % to 4 vol %.

Provided in a third aspect of the present disclosure is a sequencing library prepared by the above method. Sequencing using the sequencing library provided by the present disclosure can obtain sequencing data with higher yield and better sequencing quality.

Provided in a fourth aspect of the present disclosure is a sequencing method, comprising performing nucleic acid sequencing using the sequencing library provided in the third aspect.

Provided in a fifth aspect of the present disclosure is a single-stranded circularization reaction system, comprising oligonucleotides, DNA ligase, a ligation reaction buffer, and an additive capable of reducing DNA secondary structure and/or an additive capable of reducing DNA secondary structure and maintaining stability of single-stranded DNA.

According to an embodiment of the present disclosure, the DNA ligase comprises any one of T4 DNA ligase or Taq ligase.

According to an embodiment of the present disclosure, the single-stranded circularization sequencing reaction system further comprises a sequencing chip, the oligonucleotides being immobilized on the sequencing chip.

According to an embodiment of the present disclosure, the single-stranded circularization sequencing reaction system further comprises magnetic beads, the oligonucleotides being immobilized on the magnetic beads and/or free in a suspension of the magnetic beads.

According to an embodiment of the present disclosure, the additive capable of reducing DNA secondary structure comprises at least one of a non-ionic detergent, betaine, or DMSO.

According to an embodiment of the present disclosure, the non-ionic detergent comprises at least one of Triton-X100, Tween-20, or NP40.

According to an embodiment of the present disclosure, the additive capable of reducing DNA secondary structure and maintaining stability of single-stranded DNA comprises at least one of SSB or HSP.

According to an embodiment of the present disclosure, the additive in the single-stranded circularization reaction system comprises at least one of SSB, a non-ionic detergent, betaine, or DMSO.

According to an embodiment of the present disclosure, the additive is a mixture of a non-ionic detergent and betaine.

According to an embodiment of the present disclosure, the additive is a mixture of a non-ionic detergent and DMSO.

According to an embodiment of the present disclosure, the additive is a mixture of SSB and a non-ionic detergent and/or betaine.

Provided in a sixth aspect of the present disclosure is a rolling circle amplification reaction system for a single-stranded circular DNA library, comprising a polymerase, a rolling circle amplification reaction buffer, and an additive capable of reducing DNA secondary structure, and/or an additive capable of reducing DNA secondary structure and maintaining stability of single-stranded DNA.

According to an embodiment of the present disclosure, the polymerase comprises any one of Phi29 DNA polymerase or Bst DNA polymerase, and the polymerase can be used for rolling circle amplification.

According to a specific embodiment of the present disclosure, the rolling circle amplification reaction system further comprises a sequencing chip, the rolling circle amplification reaction being performed on the surface of the sequencing chip.

According to a specific embodiment of the present disclosure, the rolling circle amplification reaction system further comprises magnetic beads, the rolling circle amplification reaction being performed on the magnetic beads and/or in a suspension of the magnetic beads.

According to a specific embodiment of the present disclosure, the additive capable of reducing DNA secondary structure comprises at least one of a non-ionic detergent, betaine, or DMSO.

According to a specific embodiment of the present disclosure, the non-ionic detergent comprises at least one of Triton-X100, Tween-20, or NP40.

According to a specific embodiment of the present disclosure, the additive capable of reducing DNA secondary structure and maintaining stability of single-stranded DNA comprises at least one of SSB or HSP.

According to a specific embodiment of the present disclosure, the additive in the rolling circle amplification reaction system comprises at least one of SSB, or a mixture of SSB and a non-ionic detergent and/or betaine.

Provided in a seventh aspect of the present disclosure is a rolling circle amplification reaction system for a double-stranded circular DNA library, comprising a polymerase, a rolling circle amplification reaction buffer, and an additive capable of reducing DNA secondary structure and maintaining stability of single-stranded DNA, and/or an additive capable of reducing stability of the double helix structure of circular double-stranded DNA.

According to a specific embodiment of the present disclosure, the additive capable of reducing DNA secondary structure and maintaining stability of single-stranded DNA comprises at least one of SSB, or a mixture of SSB and a non-ionic detergent and/or betaine.

According to a specific embodiment of the present disclosure, the additive capable of reducing stability of the double helix structure of circular double-stranded DNA comprises at least one of formamide, ammonium sulfate, urea, or tetramethylammonium hydroxide.

According to an embodiment of the present disclosure, the polymerase comprises any one of Phi29 DNA polymerase or Bst DNA polymerase, and the polymerase can be used for rolling circle amplification.

Provided in an eighth aspect of the present disclosure is use of the method according to the first aspect, the method according to the second aspect, the sequencing library according to the third aspect, the single-stranded circularization reaction system according to the fifth aspect, the rolling circle amplification reaction system according to the sixth aspect, or the rolling circle amplification reaction system according to the seventh aspect in sequencing.

Additional aspects and advantages of the present disclosure will be partially given in the following description, partially will become apparent from the following description, or will be learned through the practice of the present disclosure.

### BRIEF DESCRIPTION OF DRAWINGS

The above and/or additional aspects and advantages of the present disclosure will become apparent and readily understandable from the description of the examples in conjunction with the accompanying drawings below.

FIG. 1 shows the GC-bias results of optimized sequencing of the methylation single-stranded circular library in Example 1 of the present disclosure.

FIG. 2 shows the GC-bias results of optimized sequencing of the methylation single-stranded circular library in Example 2 of the present disclosure.

FIG. 3 shows the GC-bias results of optimized sequencing of the methylation single-stranded circular library in Example 11 of the present disclosure.

FIG. 4 shows the GC-bias results of optimized sequencing of the methylation single-stranded circular library in Example 12 of the present disclosure.

FIG. 5 shows the GC-bias results of optimized sequencing of the methylation double-stranded circular library in Example 13 of the present disclosure.

### DESCRIPTION OF EMBODIMENTS

The embodiments of the present disclosure will be described in detail below. The embodiments described below are exemplary and are merely used to explain the present disclosure, but shall not be construed as a limitation of the present disclosure.

In addition, the terms “first” and “second” are merely used for descriptive purposes but shall not be understood as indicating or implying relative importance or implicitly indicating the quantity of indicated technical features. Thus, features defined with “first” and “second” may explicitly or implicitly include at least one such feature. In the description of the present disclosure, “a plurality of” or “more” means at least two, such as two, three, and so on, unless otherwise explicitly and specifically defined.

In the present disclosure, the term “SSB” refers to a single-stranded DNA-binding protein, which has a high affinity for single-stranded DNA (ssDNA) and can wrap around the single-stranded DNA, thereby preventing DNA degradation. Meanwhile, SSB can also maintain the single-stranded state of circular DNA and prevent single-stranded DNA from forming secondary structures.

Provided in the present disclosure is a method for preparing DNB, the method including:
(a) performing a circularization reaction on a linear DNA library to obtain a circular DNA library, wherein the circularization reaction includes a single-stranded circularization reaction or a double-stranded circularization reaction, and the circular DNA library comprises a single-stranded circular DNA library or a double-stranded circular DNA library; and
(b) performing a rolling circle amplification reaction on the circular DNA library to obtain DNB.

In the case that the circularization reaction in step (a) is a single-stranded circularization reaction,
step (a) further comprises adding at least one of the following additives to a single-stranded circularization reaction to obtain a single-stranded circular DNA library:
1) an additive capable of reducing DNA secondary structure; or
2) an additive capable of reducing DNA secondary structure and maintaining stability of single-stranded DNA, and/or
step (b) further comprises performing a rolling circle amplification reaction using the single-stranded circular DNA library from step (a), wherein at least one of the following additives is added in the rolling circle amplification reaction:
3) an additive capable of reducing DNA secondary structure; or
4) an additive capable of reducing DNA secondary structure and maintaining stability of single-stranded DNA.

In the case that the circularization reaction in step (a) is a double-stranded circularization reaction,
step (b) further comprises performing a rolling circle amplification reaction using the double-stranded circular DNA library from step (a), wherein at least one of the following additives is added in the rolling circle amplification reaction:
5) an additive capable of reducing DNA secondary structure and maintaining stability of single-stranded DNA; or
6) an additive capable of reducing stability of the double helix structure of circular double-stranded DNA.

Provided in another aspect of the present disclosure is a method for rolling circle amplification, comprising performing a rolling circle amplification reaction on a DNA library to obtain single-stranded linear DNA.

In the case that the DNA library is a single-stranded circular DNA library, at least one of the following additives is added in the rolling circle amplification reaction:
7) an additive capable of reducing DNA secondary structure; or
8) an additive capable of reducing DNA secondary structure and maintaining stability of single-stranded DNA.

In the case that the DNA library is a double-stranded circular DNA library, at least one of the following additives is added in the rolling circle amplification reaction:
9) an additive capable of reducing DNA secondary structure and maintaining stability of single-stranded DNA; or
10) an additive capable of reducing stability of the double helix structure of circular double-stranded DNA.

In response to the low circularization efficiency of circular libraries, the present disclosure significantly improves the yield of circular libraries and the coverage uniformity of regions with varying GC contents by using in the circularization system an additive that facilitates the unfolding of secondary structures. In particular, the coverage of high-GC regions in the methylation single-stranded circular library is significantly improved, which is conducive to improving the detection accuracy of methylation rates.

In particular, in response to the low data volume and poor quality in the sequencing of circular methylation libraries, the present disclosure, on the basis of optimized algorithm and sequencing script, introduces an additive during the rolling circle amplification process of circular libraries, which further significantly improves the sequencing data volume and sequencing quality of both single-stranded circular and double-stranded circular methylation libraries.

During the single-stranded circularization and rolling circle amplification of circular libraries, the double-stranded structure of DNA is unwound. The single-stranded DNA tends to form intramolecular folded secondary structures, which block the reaction binding sites of ligase and/or polymerase, resulting in reduced ligation efficiency and/or polymerization efficiency. The present disclosure introduces an additive and an additive combination capable of reducing DNA secondary structure to the circularization reaction and/or rolling circle amplification reaction of circular libraries, allowing the circularization efficiency and/or sequencing quality of circular libraries to be significantly improved. Meanwhile, an additive capable of protecting the stability of single-stranded DNA also protects some overly loose single-stranded DNA (such as methylation libraries where unmethylated Cs are converted to Us) from degradation during the reaction, achieving a significant improvement in the circularization efficiency and/or sequencing quality of circular libraries.

In particular, during the rolling circle amplification of double-stranded circular libraries, although the DNA polymerase capable of rolling circle amplification has a strand-unwinding function, some hard-to-unwind regions (such as high-GC regions) may fail to be effectively unwound. The present disclosure utilizes an additive capable of reducing stability of the double helix structure of template DNA to improve the strand-unwinding efficiency of isothermal rolling circle amplification, such that the library replication in hard-to-unwind regions is effectively initiated, thereby improving library sequencing quality.

In some specific embodiments, provided in the present disclosure is a method for improving single-stranded circularization efficiency, which specifically includes the following steps:
1) Homogenizing double-stranded DNA with TE buffer or sterile water.
2) Performing a high-temperature thermal denaturation reaction on a temperature-controlled instrument (e.g., a PCR instrument, an isothermal incubator, or a water bath) to unwind the double-stranded DNA into single-stranded DNA.
3) Immediately placing the denatured reaction mixture on ice or rapidly cooling it down to 4° C., and adding the circularization reaction mixture. The circularization reaction mixture includes a splint oligo complementary to the terminal ends of the single-stranded DNA adapter, DNA ligase (e.g., T4 DNA ligase), a circularization ligation buffer compatible with the ligase, as well as an additive capable of reducing DNA secondary structure, an additive capable of reducing DNA secondary structure and maintaining stability of single-stranded DNA, or a combination of the above two additives.
4) Performing a single-stranded circularization enzymatic reaction on a temperature-controlled instrument (e.g., a PCR instrument, an isothermal incubator, or a water bath).
5) After completion of the reaction, immediately placing the mixture on ice or rapidly cooling to down to 4° C.
6) Depending on the requirements of the sequencing application for library construction speed and library purity, optionally performing linear digestion followed by purification of the circular DNA library. Alternatively, linear digestion may be omitted, and a portion of the circularization reaction mixture may be used directly for the subsequent library amplification reaction.
7) For workflows involving linear digestion followed by purification of the circular DNA library, quantifying the concentration of the single-stranded circular library using a fluorescence-based quantification instrument and corresponding reagents, and calculating the circularization efficiency.

$$Circularization efficiency = Total amount of purified single ‐ stranded circular library / Total amount of input double ‐ stranded DNA for circularization .$$

The additive capable of reducing DNA secondary structure includes Triton-X100, Tween-20, betaine, or DMSO. The additive capable of reducing DNA secondary structure and protecting single-strand stability includes SSB or an additive or an additive combination with equivalent effects. The additive combination includes, but is not limited to, SSB+Triton-X100, SSB+Tween-20, or SSB+Triton-X100+Tween-20.

In addition to the method described in the above disclosed example, the method for improving single-stranded circularization efficiency further includes other similar modified methods. For example, in step 2), an additive capable of reducing DNA secondary structure and/or an additive capable of reducing DNA secondary structure and maintaining stability of single-stranded DNA is further added; in step 3), no further additive is added, or the above additive(s) or combinations thereof is added in both step 2) and step 3). In another modified method, the T4 DNA ligase used in step 3) is replaced with Taq DNA ligase. It should be noted that this operation requires cooling the denatured reaction mixture to the optimal reaction temperature of Taq DNA ligase (such as 45° C.).

In some specific embodiments, the technical solution of the present disclosure for improving the sequencing quality of a circular methylation library includes the following steps:
(1) Homogenizing the circular methylation library (where the circularization method includes single-stranded circularization, the above optimized single-stranded circularization method or double-stranded circularization) with Low TE buffer or sterile water.
(2) For a single-stranded circular library, adding a rolling circle amplification primer complementary to the circularization adaptor, and performing high-temperature thermal denaturation and primer annealing on a temperature-controlled instrument (e.g., PCR cycler, isothermal incubator, or a water bath); for a double-stranded circular library in which one strand is closed and the other strand contains a nick, proceeding directly to the next step without adding the primer.
(3) Adding a rolling circle replication reaction mixture, mixing and centrifuging the mixture, and performing a rolling circle amplification reaction on a temperature-controlled instrument (e.g., a PCR instrument, an isothermal incubator, or a water bath). The rolling circle replication reaction mixture for the single-stranded circular library includes a rolling circle amplification polymerase, an amplification reaction buffer compatible with the polymerase, and an additive capable of reducing DNA secondary structure and maintaining stability of single-stranded DNA. The rolling circle replication reaction mixture for the double-stranded circular library includes a rolling circle amplification polymerase, an amplification reaction buffer compatible with the polymerase, and an additive capable of reducing DNA secondary structure and maintaining stability of single-stranded DNA, and/or an additive capable of reducing stability of the double helix structure of circular double-stranded DNA.
(4) Adding a reaction-termination buffer and gently mixing the amplification product by pipetting with wide-bore pipette tips.
(5) Quantifying the concentration of single-stranded DNA using a fluorescence-based quantification instrument and corresponding quantification reagents, and calculating the sampling volume of the amplified library to be loaded onto the sequencing chip according to the requirements of the sequencing platform.
(6) Sequencing the circular methylation library using a sequencing instrument equipped with a base-imbalance-correction algorithm and an optimized sequencing script.

The additive capable of reducing DNA secondary structure and maintaining stability of single-stranded DNA includes SSB or an additive or an additive combination with equivalent effects. The additive capable of reducing DNA secondary structure includes betaine or an additive or an additive combination with equivalent effects. The additive capable of reducing stability of the double helix structure of circular double-stranded DNA includes formamide or an additive or an additive combination with equivalent effects.

In some specific embodiments, the above technical solution and modified methods for improving the circularization efficiency and sequencing quality of circular methylation libraries are also applicable to the whole-genome WGS libraries.

According to an embodiment of the present disclosure, when the working concentration of SSB is between 5 ng/μL and 130 ng/μL, the sequencing quality and data yield of the library can be greatly improved. Preferably, the working concentration of SSB is 15 ng/μL-50 ng/μL, which achieves the best effect.

According to an embodiment of the present disclosure, when the working concentration of betaine is between 0.1 M and 1 M, the coverage of high-GC regions of the library can be greatly improved. Preferably, the working concentration of betaine is 0.4 M to 0.7 M, which achieves the best effect.

According to an embodiment of the present disclosure, when the working concentration of formamide is 0.5 vol % to 4 vol %, the sequencing quality and data yield of the library can be greatly improved. Preferably, the working concentration of formamide is 2 vol % to 3 vol %, which achieves the best effect.

In the single-stranded circularization process, further adding an additive capable of reducing DNA secondary structure, or an additive or an additive combination reducing DNA secondary structure and maintaining stability of single-stranded DNA, is capable of significantly improving the yield and circularization efficiency of the single-stranded circular library. As a result, a single-stranded circular library can achieve more sequencing runs without repeated library construction, saving library construction cost and time, and further reducing the initial amount required for library construction.

In the rolling circle replication process of the circular methylation library, further adding an additive capable of reducing DNA secondary structure can reduce the adverse impact of the secondary structure (e.g., stem-loop structure, consecutive base sequence, or palindromic sequence) on rolling circle amplification efficiency, improve the uniformity of rolling circle amplification efficiency of different circular DNA molecules, and reduce the signal variations when different circular DNA molecules are amplified into DNBs, thereby improving the quality of sequencing data and reducing the coverage bias of sequencing data. In particular, after most of the Cs in the single-stranded methylation library are converted to Ts, the sequencing signal in the C channel is low, and the sequencing quality is poor, although such problems can be solved by optimization of the sequencing algorithm. However, because most of the Cs in this type of library are converted to Ts, although the probability of forming secondary structures is lower than that of conventional libraries, this loose configuration has the problem of being susceptible to degradation during the reaction process, thereby failing to produce normal DNB products and resulting in signal loss during sequencing. By introducing an additive or an additive combination capable of both unfolding DNA secondary structure and protecting loose single-stranded DNA from degradation, the present disclosure further improves the quality of sequencing data, the sequencing data yield, and the coverage uniformity of sequencing data of single-stranded circular libraries (especially methylation libraries, also including whole-genome WGS libraries). By introducing an additive or an additive combination capable of both unfolding the DNA secondary structure and protecting loose single-stranded DNA from degradation and/or an additive or an additive combination capable of reducing the stability of the double helix structure of circular double-stranded DNA, the present disclosure further improves the quality of sequencing data, the sequencing data yield, and the coverage uniformity of sequencing data of double-stranded circular libraries (especially methylation libraries, also including whole-genome WGS libraries).

As a result, a methylation library can obtain more data volume using fewer sequencing reagents, reducing the sequencing cost of methylation libraries. Meanwhile, the improvement in the coverage uniformity of sequencing data further increases the detection rate of difficult regions in methylation libraries, whole-genome WGS libraries, and other libraries to be sequenced, and enhances the accuracy of sequencing.

The additives used in the present disclosure are not limited to single agents or mixed formulations of SSB, Triton X-100, Tween-20, betaine, DMSO, NP-40, or formamide at various concentrations, nor to their cross-combinations at different concentrations. The additives may also include other agents or combinations thereof that exhibit equivalent functions, such as reducing single-stranded DNA secondary structure, or reducing single-stranded DNA secondary structure while protecting single-stranded DNA from degradation. Particularly, in the case of rolling circle amplification of double-stranded circular libraries, the additives may further include agents or combinations that produce an equivalent effect of reducing the stability of the double helix structure of circular double-stranded DNA. In addition, in steps that require reducing secondary structure interference to improve reaction efficiency and uniformity—such as the circularization step (with or without linear-DNA digestion), library replication and amplification, one-tube circularization plus amplification, and sequencing-hybridization—the circularization efficiency and sequencing quality may also be improved by fine-tuning the reaction temperature alone or in combination with different additive formulations.

The types of libraries used in the present disclosure are not limited to whole-genome WGS PCR-free, PCR libraries, whole-genome methylation libraries, or third-party whole-genome methylation libraries. They may also include targeted capture libraries, targeted methylation libraries, amplicon-type libraries (such as multiplex PCR, long-fragment PCR, Olink amplicons, immune-repertoire amplicons, 16S amplicons, or HPV amplicons), RNA libraries, stLFR libraries, and library types used in other applications.

The sequencing platforms applicable to the optimization effects of the present disclosure are not limited to the MGISEQ-2000 platform (also known as DNBSEQ-G400). Other sequencing platforms based on DNB sequencing technology, including DNBSEQ-E5, DNBSEQ-E25, DNBSEQ-G50, NBSEQ-G99, NBSEQ-G200, DNBSEQ-T7, DNBSEQ-T10, DNBSEQ-T20, as well as commercially available, developmental, or future sequencing platforms employing similar circular library sequencing principles, are also expected to benefit from the disclosed optimization.

Examples, where specific technologies or conditions are not specified, are implemented in accordance with technologies or conditions described in the literature in the art or in accordance with the product manuals. Reagents or instruments whose manufacturers are not indicated are all routine products that can be obtained commercially.

### Example 1: Optimization Experiment-1 for Improving Single-Stranded Circularization Efficiency and Coverage Uniformity of High-GC Regions in Whole-Genome Methylation Library

The reagents used in this experiment are shown in Table 1.

TABLE 1
Reagents required for the single-stranded circularization
experiment of whole-genome methylation library

| Reagent name | Brand | Cat. No. |
|---|---|---|
| MGIEasy Circularization Module | MGI | 1000005260 |
| MGIEasy DNA Purification Magnetic Bead Kit | MGI | 1000005279 |
| Tth SSB (500 ng/uL) | BGI | 1000007885 |
| Qubit ® ssDNA Assay Kit | Invitrogen | Q10212 |
| MGISEQ-2000RS High-Throughput | MGI | 1000012555 |
| Sequencing Set (PE150) | | |

The specific experimental procedure was as follows:
1) 163.8 ng of PCR product was transferred into a new 0.2 mL PCR tube, and the total volume was made up to 48 μL with TE Buffer.
2) After mixing and centrifugation, the PCR tube from the previous step was placed in a PCR instrument for reaction at 95° C. for 3 min and then 4° C. for 5 min. After the reaction was completed, the PCR tube was immediately transferred onto ice and briefly centrifuged.
3) A single-stranded circularization reaction mixture was prepared as follows: 11.6 μL/tube of Splint Buffer (a circularization buffer) and 0.5 L/tube of DNA Rapid Ligase were added to the reaction solution obtained in the previous step. No SSB was added to the control group, while 1 μL/tube or 1.5 μL/tube of the additive SSB (500 ng/μL) was further added to the experimental group. After mixing and centrifugation, the PCR tube was placed in a PCR instrument to perform the single-stranded circularization reaction at 37° C. for 30 min and then holding at 4° C.
4) After the reaction was completed, the reaction solution was briefly centrifuged, and the PCR tube was transferred onto ice to proceed to the next step of enzymatic digestion.
5) 1.4 μL/tube of Digestion Buffer and 2.6 μL/tube of Digestion Enzyme were added to the circularized product. After mixing and centrifugation, the PCR tube was placed in a PCR instrument to perform the enzymatic digestion reaction at 37° C. for 30 min and then holding at 4° C.
6) After the enzymatic digestion reaction was completed, the reaction solution was briefly centrifuged, and the PCR tube was transferred onto ice. Then, 7.5 μL of Digestion Stop Buffer was added to the PCR tube, vortexed three times for 3 s each time, and briefly centrifuged to collect the reaction solution at the bottom of the tube. The entire reaction solution was transferred into a new 1.5 mL centrifuge tube.
7) The reaction solution was purified using 170 μL of MGIEasy DNA Purification Magnetic Beads and eluted in 22 μL of TE Buffer. Then, 20 μL of the supernatant was transferred into a new 1.5 mL centrifuge tube.
8) The single-stranded circular DNA was quantified using the Qubit® ssDNA Assay Kit (a fluorescent quantification kit), and the quantification results are shown in Table 2. Compared with the control group without SSB, the circularization efficiency of the methylated single-stranded circular library with further added SSB was improved by at least 5 percentage points.

TABLE 2
Single-Stranded Circularization Results of Whole-Genome Methylation Library Before and After Optimization

| | | | Working | | | | | |
|---|---|---|---|---|---|---|---|---|
| | | | concentration | | | | | |
| | | | of Tth | | | | | |
| | | Working | SSB | | | | | |
| | | volume | further | | | | Total | |
| | | of | added to | PCR | Total | Single- | single- | |
| | | single- | single- | library | input | stranded | stranded | |
| | | stranded | stranded | main | amount | circular | circular | Single- |
| | | circularization | circularization | peak | for | library | library | stranded |
| Library | Optimization | reaction | reaction | size | circularization | concentration | amount | circularization |
| name | condition | (uL) | (ng/uL) | (bp) | (ng) | (ng/μL) | (ng) | efficiency |
| Control | No SSB | 60.1 | 0 | 434 | 163.8 | 1.87 | 41.14 | 25.1% |
| Group-1 | | | | | | | | |
| Control | | 60.1 | 0 | 434 | 163.8 | 1.71 | 37.62 | 23% |
| Group-2 | | | | | | | | |
| Experimental | 1 μL of | 61.1 | 8.18 | 434 | 163.8 | 2.35 | 51.7 | 31.6% |
| Group- | SSB | | | | | | | |
| Scheme | (500 | | | | | | | |
| 1-1 | ng/uL) | | | | | | | |
| Experimental | further | 61.1 | 8.18 | 434 | 163.8 | 2.24 | 49.28 | 30.1% |
| Group- | added | | | | | | | |
| Scheme | | | | | | | | |
| 1-2 | | | | | | | | |
| Experimental | 1.5 μL | 61.6 | 12.18 | 434 | 163.8 | 2.44 | 53.68 | 32.8% |
| Group- | of SSB | | | | | | | |
| Scheme | (500 | | | | | | | |
| 2-1 | ng/uL) | | | | | | | |
| Experimental | further | 61.6 | 12.18 | 434 | 163.8 | 2.35 | 51.7 | 31.6% |
| Group- | added | | | | | | | |
| Scheme | | | | | | | | |
| 2-2 | | | | | | | | |

The constructed whole-genome methylation single-stranded circular DNA library was subjected to DNA nanoball preparation and sequenced on an MGISEQ-2000 PE100. The sequencing procedure was performed in accordance with the standard operating procedure for MGISEQ-2000 PE100. The GC-bias analysis results of the sequencing data, as shown in FIG. 1, show that, compared with the control group with no SSB added, the optimized groups with further added 1 μl or 1.5 μl of SSB (500 ng/μL) exhibited significantly improved GC coverage uniformity.

Example 1 indicates that when the working concentration of Tth SSB added in the single-stranded circularization reaction is 8.18 to 12.18 ng/μL, the single-stranded circularization efficiency of the whole-genome methylation library can be improved, and the coverage uniformity of high-GC regions in the library can also be enhanced.

### Example 2: Optimization Experiment-2 for Improving Single-Stranded Circularization Efficiency and Coverage Uniformity of High-GC Regions in Whole-Genome Methylation Library

The reagents used in this experiment are shown in Table 3.

TABLE 3
Reagents required for the single-stranded circularization
experiment of whole-genome methylation library

| Reagent name | Brand | Cat. No. |
|---|---|---|
| MGIEasy Circularization Module | MGI | 1000005260 |
| MGIEasy DNA Purification Magnetic Bead | MGI | 1000005279 |
| Kit | | |
| Tth SSB (500 ng/uL) | BGI | 1000007885 |
| Tween-20 | BBI | A600560-0500 |
| Betaine | BBI | A600185 |
| Qubit ® ssDNA Assay Kit | Invitrogen | Q10212 |
| MGISEQ-2000RS High-Throughput | MGI | 1000012555 |
| Sequencing Set (PE150) | | |

The specific experimental procedure was as follows:
1) 300 ng of PCR product was transferred into a new 0.2 mL PCR tube, and the experiment design was performed as shown in Table 4. No additives were added to the control group, and the total volume was made up to 48 μL with TE Buffer. For Experimental Group Scheme 1, 6 μL of 5 M betaine was added, and the total volume was made up to 42 μL with TE Buffer. For Experimental Group Schemes 2 and 3, 2.5 μL of 10% Tween-20 and 6 μL of 5M betaine were added, respectively, and the total volume was made up to 39.5 μL with TE Buffer.
2) After mixing and centrifugation, the PCR tube from the previous step was placed in a PCR instrument for reaction: at 95° C. for 3 min and then 4° C. for 5 min. After the reaction was completed, the PCR tube was immediately transferred onto ice and briefly centrifuged.
3) A single-stranded circularization reaction solution was prepared as follows: 11.6 μL/tube of Splint Buffer (a circularization buffer) and 0.5 μL/tube of DNA Rapid Ligase were added to the reaction solution obtained in the previous step. Then, 2 μL/tube of the additive SSB (500 ng/μL) was added to the product of Experimental Protocol 3. After mixing and centrifugation, the PCR tube was placed in a PCR instrument to perform the single-stranded circularization reaction at 37° C. for 30 min and then holding at 4° C.
4) After the reaction was completed, the reaction solution was briefly centrifuged, and the PCR tube was transferred onto ice to proceed to the next step of enzymatic digestion.
5) 1.4 μL/tube of Digestion Buffer and 2.6 μL/tube of Digestion Enzyme were added to the circularized product. After mixing and centrifugation, the PCR tube was placed in a PCR instrument to perform the enzymatic digestion reaction at 37° C. for 30 min and then holding at 4° C.
6) After the enzymatic digestion reaction was completed, the reaction solution was briefly centrifuged, and the PCR tube was transferred onto ice. Then, 7.5 μL of Digestion Stop Buffer was added to the PCR tube, vortexed three times for 3 s each time, and briefly centrifuged to collect the reaction solution at the bottom of the tube. The entire reaction solution was transferred into a new 1.5 mL centrifuge tube.
7) The reaction solution was purified using 170 μL of MGIEasy DNA Purification Magnetic Beads and eluted in 22 μL of TE Buffer. Then, 20 μL of the supernatant was transferred into a new 1.5 mL centrifuge tube.
8) The single-stranded circular DNA was quantified using the Qubit® ssDNA Assay Kit (a fluorescent quantification kit), and the quantification results are shown in Table 4. Compared with the control group with no further reagents added, the experimental groups with further added betaine, betaine+Tween-20, and betaine+Tween-20+SSB all exhibited a certain improvement in the single-stranded circularization efficiency of the whole-genome methylation library.

TABLE 4
Single-Stranded Circularization Results of Whole-Genome Methylation Library Before and After Optimization

| | | | Working | | | | | |
|---|---|---|---|---|---|---|---|---|
| | | Working | concentration | | | | | |
| | | volume | of additive | | | | Total | |
| | | of | further | PCR | Total | Single- | single- | |
| | | single- | added to | library | input | stranded | stranded | |
| | | stranded | single- | main | amount | circular | circular | Single- |
| | | circularization | stranded | peak | for | library | library | stranded |
| Library | Optimization | reaction | circularization | size | circularization | concentration | amount | circularization |
| name | condition | (uL) | reaction | (bp) | (ng) | (ng/μL) | (ng) | efficiency |
| Control | No | 60.1 | 0 | 434 | 300 | 2.34 | 51.48 | 17.2% |
| Group-1 | further | | | | | | | |
| | reagents | | | | | | | |
| | added | | | | | | | |
| Experimental | 6 μL of | 60.1 | 0.50M | 434 | 300 | 2.59 | 56.98 | 19% |
| Group- | 5M | | betaine | | | | | |
| Scheme 1 | betaine | | | | | | | |
| | further | | | | | | | |
| | added | | | | | | | |
| Experimental | 2.5 μL | 60.1 | 0.42% | 434 | 300 | 2.83 | 62.26 | 20.8% |
| Group- | of 10% | | Tween- | | | | | |
| Scheme | Tween- | | 20 + | | | | | |
| 2-1 | 20 and | | 0.50M | | | | | |
| Experimental | 6 μL of | 60.1 | betaine | 434 | 300 | 2.73 | 60.06 | 20% |
| Group- | 5M | | | | | | | |
| Scheme | betaine | | | | | | | |
| 2-2 | further | | | | | | | |
| | added | | | | | | | |
| Experimental | 2.5 μL | 62.1 | 0.40% | 434 | 300 | 3.21 | 70.62 | 23.5% |
| Group- | of 10% | | Tween- | | | | | |
| Scheme | Tween- | | 20 + | | | | | |
| 3-1 | 20, 6 | | 0.48M | | | | | |
| Experimental | μL of | 62.1 | betaine + | 434 | 300 | 3.09 | 67.98 | 22.7% |
| Group- | 5M | | 16.10 | | | | | |
| Scheme | betaine, | | ng/uL | | | | | |
| 3-2 | and 2 | | Tth SSB | | | | | |
| | μL of | | | | | | | |
| | Tth | | | | | | | |
| | SSB | | | | | | | |
| | (500 | | | | | | | |
| | ng/uL) | | | | | | | |
| | further | | | | | | | |
| | added | | | | | | | |

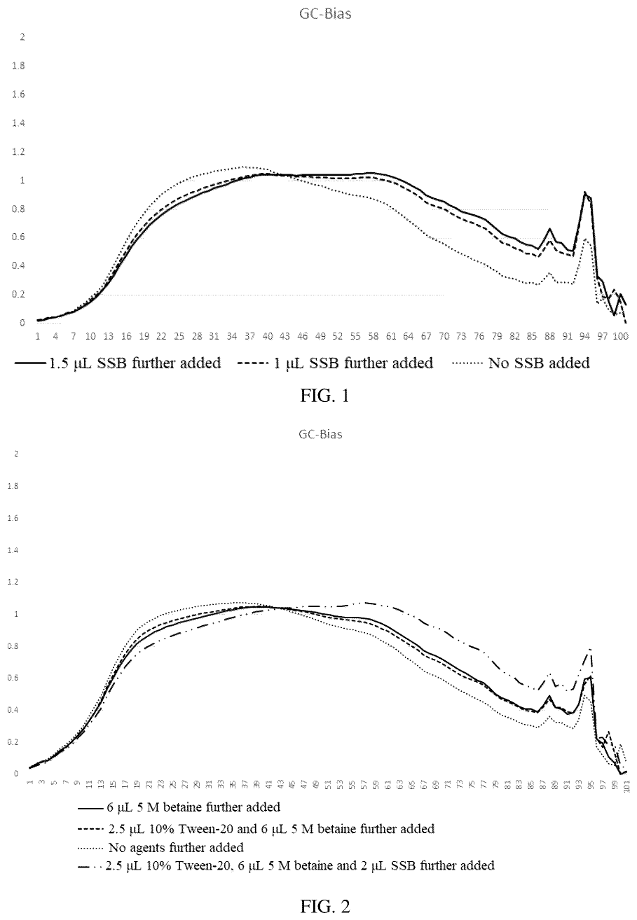

The constructed whole-genome methylation single-stranded circular DNA library was subjected to DNA nanoball preparation and sequenced on an MGISEQ-2000 PE100. The sequencing procedure was performed in accordance with the standard operating procedure for MGISEQ-2000 PE100. The GC-bias analysis results of the sequencing data, as shown in FIG. 2, show that, compared with the control group with no further reagents added, the optimized groups with further added betaine, Tween-20+betaine, and Tween-20+betaine+SSB all exhibited improved GC coverage uniformity.

Example 2 indicates that when the working concentrations of the combined formulations further added in the single-stranded circularization reaction are 0.48 to 0.50M betaine, 0.40% to 0.42% Tween-20, and 16.10 ng/μL Tth SSB, the single-stranded circularization efficiency of the whole-genome methylation library can be improved, and the coverage uniformity of high-GC regions in the library can also be enhanced, among which Tth SSB has the relatively largest contribution to improving the high-GC coverage.

### Example 3: Optimization Experiment-3 for Improving Single-Stranded Circularization Efficiency of Whole-Genome Methylation Library

The reagents used in this experiment are shown in Table 5.

TABLE 5
Reagents required for the single-stranded circularization
experiment of whole-genome methylation library

| Reagent name | Brand | Cat. No. |
|---|---|---|
| MGIEasy Circularization Module | MGI | 1000005260 |
| MGIEasy DNA Purification Magnetic Bead | MGI | 1000005279 |
| Kit | | |
| DMSO | Sigma | D2650 |
| Triton X-100 | Diamond | A110694-0500 |
| Tween-20 | BBI | A600560-0500 |
| Qubit ® ssDNA Assay Kit | Invitrogen | Q10212 |

The specific experimental procedure was as follows:
1) 200 ng of PCR product was transferred into a new 0.2 mL PCR tube, and various additives were added in accordance with Table 6. The volume of PCR products with Test Nos. 1 to 4 was made up to 48 μL with TE Buffer.
2) After mixing and centrifugation, the PCR tube from the previous step was placed in a PCR instrument for reaction at 95° C. for 3 min and then 4° C. for 5 min. After the reaction was completed, the PCR tube was immediately transferred onto ice and briefly centrifuged.
3) A single-stranded circularization reaction solution was prepared as follows: 11.6 μL/tube of Splint Buffer (a circularization buffer) and 0.5 μL/tube of DNA Rapid Ligase were added to the reaction solution obtained in the previous step. After mixing and centrifugation, the PCR tube was placed in a PCR instrument to perform the single-stranded circularization reaction at 37° C. for 30 min and then holding at 4° C.
4) After the reaction was completed, the reaction solution was briefly centrifuged, and the PCR tube was transferred onto ice to proceed to the next step of enzymatic digestion.
5) 1.4 μL/tube of Digestion Buffer and 2.6 μL/tube of Digestion Enzyme were added to the circularized product. After mixing and centrifugation, the PCR tube was placed in a PCR instrument to perform the enzymatic digestion reaction at 37° C. for 30 min and then holding at 4° C.
6) After the enzymatic digestion reaction was completed, the reaction solution was briefly centrifuged, and the PCR tube was transferred onto ice. Then, 7.5 μL of Digestion Stop Buffer was added to the PCR tube, vortexed three times for 3 s each time, and briefly centrifuged to collect the reaction solution at the bottom of the tube. The entire reaction solution was transferred into a new 1.5 mL centrifuge tube.
7) The reaction solution was purified using 170 μL of MGIEasy DNA Purification Magnetic Beads and eluted in 27 μL of TE Buffer. Then, 25 μL of the supernatant was transferred into a new 1.5 mL centrifuge tube.
8) The single-stranded circular DNA was quantified using the Qubit® ssDNA Assay Kit (a fluorescent quantification kit), and the quantification results are shown in Table 6. Compared with the control group, the experimental groups with different additive test schemes all exhibited a certain improvement in the concentration and circularization efficiency of the methylated single-stranded circular library, among which the improvement effect of Experimental Group Scheme 3 was relatively more significant.

TABLE 6
Single-Stranded Circularization Results of Whole-Genome
Methylation Library Before and After Optimization

| | | Additive | Working | | | |
|---|---|---|---|---|---|---|
| | | further | volume of | | Single- | |
| | | added to | single- | | stranded | |
| | | single- | stranded | Working | circular | Single- |
| | | stranded | circularization | concentration | library | stranded |
| Test | Library | circularization | reaction | of additive | concentration | circularization |
| No. | name | reaction | (uL) | added | (ng/μL) | efficiency |
| 1 | Control | None | 60.1 | 0 | 1.3 | 11.70% |
| | group | | | | | |
| 2 | Experimental | 1.5 μL of | 60.1 | 2.50% | 1.52 | 13.68% |
| | Group- | 100% | | DMSO | | |
| | Scheme 1 | DMSO | | | | |
| 3 | Experimental | 2.5 μL of | 60.1 | 0.83% | 1.88 | 16.92% |
| | Group- | 20% Triton | | Triton X- | | |
| | Scheme 2 | X-100 | | 100 | | |
| 4 | Experimental | 2.5 μL of | 60.1 | 0.42% | 2.35 | 21.15% |
| | Group- | 10% Tween- | | Tween-20 | | |
| | Scheme 3 | 20 | | | | |

Example 3 indicates that when the working concentrations of the additive or additive combination further added in the single-stranded circularization reaction are 2.50% DMSO, 0.83% Triton X-100, and 0.42% Tween-20, the single-stranded circularization efficiency of the whole-genome methylation library can be improved.

### Example 4: Optimization Experiment-1 for Improving Single-Stranded Circularization Efficiency of Whole-Genome PCR-Free Library

The reagents used in this experiment are shown in Table 7.

TABLE 7
Reagents required for the single-stranded circularization
experiment of whole-genome PCR-free library

| Reagent name | Brand | Cat. No. |
|---|---|---|
| MGIEasy Circularization Module | MGI | 1000005260 |
| MGIEasy DNA Purification | MGI | 1000005279 |
| Magnetic Bead Kit | | |
| Tth SSB (500 ng/uL) | BGI | 1000007885 |
| Qubit ® ssDNA Assay Kit | Invitrogen | Q10212 |

The specific experimental procedure was as follows:
1) 150 ng of whole-genome PCR-free ligation product (after fragmentation and double selection) was transferred into a new 0.2 mL PCR tube, and the total volume was made up to 48 μL with TE Buffer.
2) After mixing and centrifugation, the PCR tube from the previous step was placed in a PCR instrument for reaction at 95° C. for 3 min. After the reaction was completed, the PCR tube was immediately transferred onto ice and briefly centrifuged.
3) A single-stranded circularization reaction solution was prepared as follows: 11.6 μL/tube of Splint Buffer (a circularization buffer) and 0.5 μL/tube of DNA Rapid Ligase were added to the reaction solution obtained in the previous step. As shown in Table 8, 1 μL/tube, 1.5 μL/tube, or 2 μL/tube of the additive SSB (500 ng/μL) was added to the experimental group. After mixing and centrifugation, the PCR tube was placed in a PCR instrument to perform the single-stranded circularization reaction at 37° C. for 30 min and then holding at 4° C.
4) After the reaction was completed, the reaction solution was briefly centrifuged, and the PCR tube was transferred onto ice to proceed to the next step of enzymatic digestion.
5) 1.4 μL/tube of Digestion Buffer and 2.6 μL/tube of Digestion Enzyme were added to the circularized product. After mixing and centrifugation, the PCR tube was placed in a PCR instrument to perform the enzymatic digestion reaction at 37° C. for 30 min and then holding at 4° C.
6) After the enzymatic digestion reaction was completed, the reaction solution was briefly centrifuged, and the PCR tube was transferred onto ice. Then, 7.5 μL of Digestion Stop Buffer was added to the PCR tube, vortexed three times for 3 s each time, and briefly centrifuged to collect the reaction solution at the bottom of the tube. The entire reaction solution was transferred into a new 1.5 mL centrifuge tube.
7) The reaction solution was purified using 170 μL of MGIEasy DNA Purification Magnetic Beads and eluted in 22 μL of TE Buffer. Then, 20 μL of the supernatant was transferred into a new 1.5 mL centrifuge tube.
8) The single-stranded circular DNA was quantified using the Qubit® ssDNA Assay Kit (a fluorescent quantification kit), and the quantification results are shown in Table 8. Compared with the control group with no SSB added, the concentration and circularization efficiency of the whole-genome PCR-free single-stranded circular libraries further added with different volumes of SSB were significantly improved by approximately 10%. The further addition of 1 μL of SSB (500 ng/μL) can achieve a relatively ideal effect.

TABLE 8
Single-Stranded Circularization Results of Whole-Genome PCR-Free Library Before and After Optimization

| | | | Working | | | | | |
|---|---|---|---|---|---|---|---|---|
| | | | concentration | | | | | |
| | | | of Tth | | | | | |
| | | | SSB | | | | | |
| | | Working | further | PCR- | | | Total | |
| | | volume of | added to | free | Total | Single- | single- | |
| | | single- | single- | library | input | stranded | stranded | |
| | | stranded | stranded | main | amount | circular | circular | Single- |
| | | circularization | circularization | peak | for | library | library | stranded |
| Library | | reaction | reaction | size | circularization | concentration | amount | circularization |
| name | Additive | (uL) | (ng/uL) | (bp) | (ng) | (ng/μL) | (ng) | efficiency |
| Control | None | 60.1 | 0 | 580 | 150 | 1.36 | 29.92 | 19.9% |
| Group-1 | | | | | | | | |
| Control | | | | 580 | 150 | 1.48 | 32.56 | 21.7% |
| Group-2 | | | | | | | | |
| Experimental | 1 μL | 61.1 | 8.18 | 580 | 150 | 2.26 | 49.72 | 33.1% |
| Group- | of | | | | | | | |
| Scheme | SSB | | | | | | | |
| 1-1 | (500 | | | | | | | |
| Experimental | ng/uL) | | | 580 | 150 | 2.21 | 48.62 | 32.4% |
| Group- | further | | | | | | | |
| Scheme | added | | | | | | | |
| 1-2 | | | | | | | | |
| Experimental | 1.5 | 61.6 | 12.18 | 580 | 150 | 2.33 | 51.26 | 34.2% |
| Group- | μL of | | | | | | | |
| Scheme | SSB | | | | | | | |
| 2-1 | (500 | | | | | | | |
| Experimental | ng/uL) | | | 580 | 150 | 2.08 | 45.76 | 30.5% |
| Group- | further | | | | | | | |
| Scheme | added | | | | | | | |
| 2-2 | | | | | | | | |
| Experimental | 2 μL | 62.1 | 16.10 | 580 | 150 | 2.02 | 44.44 | 29.6% |
| Group- | of | | | | | | | |
| Scheme | SSB | | | | | | | |
| 3-1 | (500 | | | | | | | |
| Experimental | ng/uL) | | | 580 | 150 | 1.83 | 40.26 | 26.8% |
| Group- | further | | | | | | | |
| Scheme | added | | | | | | | |
| 3-2 | | | | | | | | |

Example 4 indicates that when the working concentration of Tth SSB further added in the single-stranded circularization reaction is 8.18 to 16.10 ng/μL, the single-stranded circularization efficiency of the whole-genome PCR-free library can be significantly improved, and it reaches saturation at the working concentration of Tth SSB of 8.18 ng/μL.

### Example 5: Optimization Experiment-2 for Improving Single-Stranded Circularization Efficiency of Whole-Genome PCR-Free Library

The reagents used in this experiment are shown in Table 9.

TABLE 9
Reagents required for the single-stranded circularization
experiment of whole-genome PCR-free library

| Reagent name | Brand | Cat. No. |
|---|---|---|
| MGIEasy Circularization Module | MGI | 1000005260 |
| MGIEasy DNA Purification | MGI | 1000005279 |
| Magnetic Bead Kit | | |
| Tween-20 | BBI | A600560-0500 |
| Triton-X100 | Diamond | A110694-0500 |
| NP40 | Diamond | A100109-0100 |
| Qubit ® ssDNA Assay Kit | Invitrogen | Q10212 |

The specific experimental procedure was as follows:
1) 96.6 ng of whole-genome PCR-free ligation product (after fragmentation and single selection) was transferred into a new 0.2 mL PCR tube. As shown in Table 10, no additives were added to the control group, and the total volume was made up to 48 μL with TE Buffer. For Experimental Group Schemes 1 and 2, 2.5 μL and 5 μL of 10% Tween-20 were further added, respectively, and the total volume was made up to 45.5 μL and 43 μL with TE Buffer, respectively. For Experimental Group Schemes 3 and 4, 2.5 μL and 5 μL of 20% Triton X-100 were further added, respectively, and the total volume was made up to 45.5 μL and 43 μL with TE Buffer, respectively. For Experimental Group Schemes 5 and 6, 2.5 μL and 5 μL of 10% NP40 were further added, respectively, and the total volume was made up to 45.5 μL and 43 μL with TE Buffer, respectively.
2) After mixing and centrifugation, the PCR tube from the previous step was placed in a PCR instrument for reaction at 95° C. for 3 min and then 4° C. for 5 min. After the reaction was completed, the PCR tube was immediately transferred onto ice and briefly centrifuged.
3) A single-stranded circularization reaction solution was prepared as follows: 11.6 μL/tube of Splint Buffer (a circularization buffer) and 0.5 L/tube of DNA Rapid Ligase were added to the reaction solution obtained in the previous step. After mixing and centrifugation, the PCR tube was placed in a PCR instrument to perform the single-stranded circularization reaction at 37° C. for 30 min and then holding at 4° C.
4) After the reaction was completed, the reaction solution was briefly centrifuged, and the PCR tube was transferred onto ice to proceed to the next step of enzymatic digestion.
5) 1.4 μL/tube of Digestion Buffer and 2.6 μL/tube of Digestion Enzyme were added to the circularized product. After mixing and centrifugation, the PCR tube was placed in a PCR instrument to perform the enzymatic digestion reaction at 37° C. for 30 min and then holding at 4° C.
6) After the enzymatic digestion reaction was completed, the reaction solution was briefly centrifuged, and the PCR tube was transferred onto ice. Then, 7.5 μL of Digestion Stop Buffer was added to the PCR tube, vortexed three times for 3 s each time, and briefly centrifuged to collect the reaction solution at the bottom of the tube. The entire reaction solution was transferred into a new 1.5 mL centrifuge tube.
7) The reaction solution was purified using 170 μL of MGIEasy DNA Purification Magnetic Beads and eluted in 22 μL of TE Buffer. Then, 20 μL of the supernatant was transferred into a new 1.5 mL centrifuge tube.
8) The single-stranded circular DNA was quantified using the Qubit® ssDNA Assay Kit (a fluorescent quantification kit), and the quantification results are shown in Table 10. Compared with the control group with no further reagents added, the experimental groups with further added Tween-20, Triton-X100, and NP40 all exhibited a certain improvement in the single-stranded circularization concentration and circularization efficiency of the whole-genome methylation library.

TABLE 10
Single-Stranded Circularization Results of Whole-Genome PCR-Free Library Before and After Optimization

| | | | Working | | | | | |
|---|---|---|---|---|---|---|---|---|
| | | | concentration | | | | | |
| | | Working | of additive | | | | Total | |
| | | volume of | further | PCR | Total | Single- | single- | |
| | | single- | added to | library | input | stranded | stranded | |
| | | stranded | single- | main | amount | circular | circular | Single- |
| | | circularization | stranded | peak | for | library | library | stranded |
| Library | Optimization | reaction | circularization | size | circularization | concentration | amount | circularization |
| name | condition | (uL) | reaction | (bp) | (ng) | (ng/μL) | (ng) | efficiency |
| Control | No | 60.1 | 0 | 850 | 96.6 | 1.07 | 16.05 | 16.61% |
| Group-1 | further | | | | | | | |
| | reagents | | | | | | | |
| Experimental | 2.5 μL of | 60.1 | 0.42% | 850 | 96.6 | 1.41 | 21.15 | 21.89% |
| Group- | 10% | | Tween-20 | | | | | |
| Scheme 1 | Tween- | | | | | | | |
| | 20 | | | | | | | |
| | further | | | | | | | |
| | added | | | | | | | |
| Experimental | 5 μL of | 60.1 | 0.84% | 850 | 96.6 | 1.39 | 20.85 | 21.58% |
| Group- | 10% | | Tween-20 | | | | | |
| Scheme 2 | Tween- | | | | | | | |
| | 20 | | | | | | | |
| | further | | | | | | | |
| | added | | | | | | | |
| Experimental | 2.5 μL of | 60.1 | 0.42% | 850 | 96.6 | 1.22 | 18.3 | 18.94% |
| Group- | 20% | | Triton- | | | | | |
| Scheme 3 | Triton- | | X100 | | | | | |
| | X100 | | | | | | | |
| | further | | | | | | | |
| | added | | | | | | | |
| Experimental | 5 μL of | 60.1 | 0.84% | 850 | 96.6 | 1.46 | 21.9 | 22.67% |
| Group- | 20% | | Triton- | | | | | |
| Scheme 4 | Triton- | | X100 | | | | | |
| | X100 | | | | | | | |
| | further | | | | | | | |
| | added | | | | | | | |
| Experimental | 2.5 μL of | 60.1 | 0.42% | 850 | 96.6 | 1.23 | 18.45 | 19.10% |
| Group- | 10% | | NP40 | | | | | |
| Scheme 5 | NP40 | | | | | | | |
| | further | | | | | | | |
| | added | | | | | | | |
| Experimental | 5 μL of | 60.1 | 0.84% | 850 | 96.6 | 1.45 | 21.75 | 22.52% |
| Group- | 10% | | NP40 | | | | | |
| Scheme 6 | NP40 | | | | | | | |
| | further | | | | | | | |
| | added | | | | | | | |

Example 5 indicates that when the working concentration of Tween-20, Triton-X100, and NP40 further added in the single-stranded circularization reaction is 0.42 to 0.84%, the single-stranded circularization efficiency of the whole-genome PCR-free library can be relatively significantly improved.

### Example 6: Optimization Experiment-2 for Improving Single-Stranded Circularization Efficiency of Whole-Genome DNA Library (PCR-Based Library Construction Method)

The reagents used in this experiment are shown in Table 11.

TABLE 11
Reagents required for the single-stranded circularization
experiment of whole-genome DNA library (PCR-
based library construction method)

| Reagent name | Brand | Cat. No. |
|---|---|---|
| MGIEasy Circularization Module | MGI | 1000005260 |
| MGIEasy DNA Purification | MGI | 1000005279 |
| Magnetic Bead Kit | | |
| Tween-20 | BBI | A600560-0500 |
| Triton-X100 | Diamond | A110694-0500 |
| NP40 | Diamond | A100109-0100 |
| Qubit ® ssDNA Assay Kit | Invitrogen | Q10212 |

The specific experimental procedure was as follows:
1) 155.5 ng of WGS PCR product (dual barcode) was transferred into a new 0.2 mL PCR tube. As shown in Table 12, no additives were added to the control group, and the total volume was made up to 48 μL with TE Buffer. For Experimental Group Schemes 1, 2, and 3, 2.5 μL of 10% Tween-20, 2.5 μL of 20% Triton X-100, and 2.5 μL of 10% NP40 were further added, respectively, and the total volume was made up to 45.5 μL with TE Buffer for each.
2) After mixing and centrifugation, the PCR tube from the previous step was placed in a PCR instrument for reaction at 95° C. for 3 min and then 4° C. for 5 min. After the reaction was completed, the PCR tube was immediately transferred onto ice and briefly centrifuged.
3) A single-stranded circularization reaction solution was prepared as follows: 11.6 μL/tube of Splint Buffer (a circularization buffer) and 0.5 L/tube of DNA Rapid Ligase were added to the reaction solution obtained in the previous step. After mixing and centrifugation, the PCR tube was placed in a PCR instrument to perform the single-stranded circularization reaction at 37° C. for 30 min and then holding at 4° C.
4) After the reaction was completed, the reaction solution was briefly centrifuged, and the PCR tube was transferred onto ice to proceed to the next step of enzymatic digestion.
5) 1.4 μL/tube of Digestion Buffer and 2.6 μL/tube of Digestion Enzyme were added to the circularized product. After mixing and centrifugation, the PCR tube was placed in a PCR instrument to perform the enzymatic digestion reaction at 37° C. for 30 min and then holding at 4° C.
6) After the enzymatic digestion reaction was completed, the reaction solution was briefly centrifuged, and the PCR tube was transferred onto ice. Then, 7.5 μL of Digestion Stop Buffer was added to the PCR tube, vortexed three times for 3 s each time, and briefly centrifuged to collect the reaction solution at the bottom of the tube. The entire reaction solution was transferred into a new 1.5 mL centrifuge tube.
7) The reaction solution was purified using 170 μL of MGIEasy DNA Purification Magnetic Beads and eluted in 22 μL of TE Buffer. Then, 20 μL of the supernatant was transferred into a new 1.5 mL centrifuge tube.
8) The single-stranded circular DNA was quantified using the Qubit® ssDNA Assay Kit (a fluorescent quantification kit), and the quantification results are shown in Table 12. Compared with the control group with no further reagents added, the experimental groups with further added Tween-20, Triton-X100, and NP40 all exhibited a certain improvement in the single-stranded circularization concentration and circularization efficiency of the whole-genome methylation library.

TABLE 12
Single-Stranded Circularization Results of Whole-Genome DNA Library
(PCR-Based Library Construction Method) Before and After Optimization

| | | | Working | | | | | |
|---|---|---|---|---|---|---|---|---|
| | | Working | concentration | | | | | |
| | | volume | of additive | | | | Total | |
| | | of | further | PCR | Total | Single- | single- | |
| | | single- | added to | library | input | stranded | stranded | |
| | | stranded | single- | main | amount | circular | circular | Single- |
| | | circularization | stranded | peak | for | library | library | stranded |
| Library | Optimization | reaction | circularization | size | circularization | concentration | amount | circularization |
| name | condition | (uL) | reaction | (bp) | (ng) | (ng/μL) | (ng) | efficiency |
| Control | No | 60.1 | 0 | 500 | 155.5 | 0.938 | 14.07 | 9.05% |
| group | further | | | | | | | |
| | reagents | | | | | | | |
| Experimental | 2.5 μL | 60.1 | 0.42% | 500 | 155.5 | 1.29 | 19.35 | 12.44% |
| Group- | of 10% | | Tween- | | | | | |
| Scheme 1 | Tween- | | 20 | | | | | |
| | 20 | | | | | | | |
| | further | | | | | | | |
| | added | | | | | | | |
| Experimental | 2.5 μL | 60.1 | 0.42% | 500 | 155.5 | 1.12 | 16.8 | 10.80% |
| Group- | of 20% | | Triton- | | | | | |
| Scheme 2 | Triton- | | X100 | | | | | |
| | X100 | | | | | | | |
| | further | | | | | | | |
| | added | | | | | | | |
| Experimental | 2.5 μL | 60.1 | 0.42% | 500 | 155.5 | 1.11 | 16.65 | 10.71% |
| Group- | of 10% | | NP40 | | | | | |
| Scheme 3 | NP40 | | | | | | | |
| | further | | | | | | | |
| | added | | | | | | | |

Example 6 indicates that when the working concentration of Tween-20, Triton-X100, and NP40 further added in the single-stranded circularization reaction is 0.42%, the single-stranded circularization efficiency of the whole-genome DNA library (dual barcode) can be slightly improved.

### Example 7: Optimization Experiment for Improving Sequencing Quality of Whole-Genome Methylation Double-Stranded Circular Library

The reagents used in this experiment are shown in Table 13.

TABLE 13
Reagents required for DNB preparation and sequencing of
whole-genome methylation double-stranded circular library

| Reagent name | Brand | Cat. No. |
|---|---|---|
| DNBSEQ DNB Preparation Kit | MGI | 1000016115 |
| (included in the subsequent | | |
| sequencing kit set) | | |
| WGBS DNB Buffer | MGI | 940-000308-00 |
| Tth SSB (2 ug/uL) | BGI | 1000007886 |
| Qubit ® ssDNA Assay Kit | Invitrogen | Q10212 |
| MGISEQ-2000RS High-throughput | MGI | 1000012555 |
| Sequencing Set (PE150) | | |

The specific experimental procedure was as follows:
1) 26.4 ng of whole-genome methylation double-stranded circular library was added to a new 0.2 mL PCR tube, and the volume was made up to 10 μL with Low TE Buffer, followed by the sequential addition of the following reagents on ice: 10 μL of WGBS DNB Buffer, 20 μL of DNB polymerase mixture I, and 2 μL of DNB polymerase mixture II (LC). As shown in Table 14, X μL of SSB (2 μg/μL) (where X can be 0.5 μL, 1 μL, 1.5 μL, or 2 μL) was further added to the experimental group.
2) After mixing and centrifugation, the DNB preparation reaction was performed using a PCR instrument at 30° C. for 15 min and then holding at 4° C.
3) After the temperature of the PCR instrument reached 4° C., 10 μL of DNB stop buffer was immediately added. The mixture was gently mixed by pipetting 5-8 times with wide-bore pipette tips, with no shaking or vigorous pipetting allowed, and stored at 4° C. for later use.
4) The single-stranded circular DNA was quantified using the Qubit® ssDNA Assay Kit (a fluorescent quantification kit).
5) Then, 450 ng of DNB was loaded onto the chip and sequenced using the optimized version of MGISEQ-2000 PE150 (with optimized sequencing algorithm and sequencing script).
6) The sequencing results are shown in Table 14. With the increase in the volume of Tth SSB (2 μg/μL) further added, both the Total Reads yield and Total Q30% of the sequencing were improved, and were close to the sequencing data volume and Q30% of the PCR-free control library.

TABLE 14
DNB Preparation Optimized Sequencing Results of Whole-Genome
Methylation Double-Stranded Circular Library Using Optimized
Version of MGISEQ-2000 PE150 (With Optimized Base Imbalance
Sequencing Algorithm and Corresponding Sequencing Script)

| | | Volume of | | | | | |
|---|---|---|---|---|---|---|---|
| | | Tth SSB | | | | | |
| | | (2 ug/uL) | | Working | | | |
| | | further | | volume of | Working | | |
| | | added to | | DNB | concentration | | |
| | | DNB | | preparation | of Tth SSB | Total | Total |
| Experimental | Library | preparation | | reaction | further added | Reads | Q30 |
| design | name | reaction | | (uL) | (ng/uL) | (M) | (%) |
| Control | Control | 0 | μL | 42 | 0 | 320.86 | 89.35 |
| group before | group | | | | | | |
| WGBS | | | | | | | |
| optimization | | | | | | | |
| (negative | | | | | | | |
| control | | | | | | | |
| group) | | | | | | | |
| Experimental | Double- | 0.5 | μL | 42.5 | 23.53 | 359.36 | 91.53 |
| Group- | stranded | | | | | | |
| Scheme 1 | circular | | | | | | |
| after WGBS | library 1 | | | | | | |
| optimization | | | | | | | |
| Experimental | Double- | 1 | μL | 43 | 46.51 | 385.79 | 92.55 |
| Group- | stranded | | | | | | |
| Scheme 2 | circular | | | | | | |
| after WGBS | library 2 | | | | | | |
| optimization | | | | | | | |
| Experimental | Double- | 1.5 | μL | 43.5 | 68.97 | 389.3 | 92.83 |
| Group- | stranded | | | | | | |
| Scheme 3 | circular | | | | | | |
| after WGBS | library 3 | | | | | | |
| optimization | | | | | | | |
| Experimental | Double- | 2 | μL | 44 | 90.91 | 419.29 | 93.99 |
| Group- | stranded | | | | | | |
| Scheme 4 | circular | | | | | | |
| after WGBS | library 4 | | | | | | |
| optimization | | | | | | | |
| WGS library | PCR | 0 | μL | 42 | 0 | 422.95 | 94.68 |
| control group | Free | | | | | | |
| (positive | library | | | | | | |
| control | | | | | | | |
| group) | | | | | | | |

Example 7 indicates that when the working concentration of Tth SSB further added to the DNB preparation reaction is 23.53 to 90.91 ng/μL, the use of MGISEQ-2000 sequencing optimized with the base imbalance sequencing algorithm and the corresponding sequencing script can significantly improve the sequencing quality and the sequencing data yield of the whole-genome methylation double-stranded circular library.

### Example 8: Optimization Experiment for Improving Sequencing Quality of Whole-Genome Methylation Single-Stranded Circular Library

The reagents used in this experiment are shown in Table 15.

TABLE 15
Reagents required for DNB preparation and sequencing of
whole-genome methylation single-stranded circular library

| Reagent name | Brand | Cat. No. |
|---|---|---|
| DNBSEQ DNB Preparation Kit | MGI | 1000016115 |
| (included in the subsequent | | |
| sequencing kit set) | | |
| Tth SSB (2 ug/uL) | BGI | 1000007886 |
| Qubit ® ssDNA Assay Kit | Invitrogen | Q10212 |
| MGISEQ-2000RS High-throughput | MGI | 1000012555 |
| Sequencing Set (PE150) | | |

The specific experimental procedure was as follows:
1) 12 ng of whole-genome methylation single-stranded circular library was added to a new 0.2 mL PCR tube, and the volume was made up to 10 μL with Low TE Buffer, followed by the addition of 10 μL of DNB preparation buffer.
2) After mixing and centrifugation, denaturation and annealing reactions were performed using a PCR instrument at 95° C. for 1 min, 65° C. for 1 min, 40° C. for 1 min, and then holding at 4° C.
3) After the reaction was completed, the reaction solution was briefly centrifuged, and the following reagents were sequentially added to the PCR tube from the previous step on ice: 20 μL of DNB polymerase mixture I and 2 μL of DNB polymerase mixture II (LC). As shown in Table 16, 1 μL of SSB (2 μg/μL) was further added to the experimental group.
4) After mixing and centrifugation, the DNB preparation reaction was performed using a PCR instrument at 30° C. for 15 min and then holding at 4° C.
5) After the temperature of the PCR instrument reached 4° C., 10 μL of DNB stop buffer was immediately added. The mixture was gently mixed by pipetting 5-8 times with wide-bore pipette tips, with no shaking or vigorous pipetting allowed, and stored at 4° C. for later use.
6) The single-stranded circular DNA was quantified using the Qubit® ssDNA Assay Kit (a fluorescent quantification kit).
7) Then, 450 ng/lane of DNB was loaded onto the chip and sequenced using the optimized version of MGISEQ-2000 PE150 (with optimized sequencing algorithm and sequencing script).
8) The sequencing results are shown in Table 16. The sequencing quality, Total Reads yield, and Total Q30% of the methylation library with 1 μL of SSB (2 ug/uL) further added to the DNB preparation reaction were significantly improved compared with the control group without addition, and were close to the sequencing data volume and Q30% of the WGS PCR Free control library.

TABLE 16
DNB Preparation Optimized Sequencing Results of Whole-Genome Methylation
Single-Stranded Circular Library Using Optimized Version of MGISEQ-2000
PE150 (With Optimized Sequencing Algorithm and Sequencing Script)

| | | Volume of | | | | |
|---|---|---|---|---|---|---|
| | | Tth SSB | | | | |
| | | (2 ug/uL) | Working | | | |
| | | further | volume of | Working | | |
| | | added to | DNB | concentration | | |
| | | DNB | preparation | of Tth SSB | Total | Total |
| Experimental | Library | preparation | reaction | further added | Reads | Q30 |
| design | name | reaction | (uL) | (ng/uL) | (M) | (%) |
| Control | Control | 0 μL | 42 | 0 | 396.16 | 90.13 |
| group before | Group 1 | | | | | |
| WGBS | Control | 0 μL | | | 397.41 | 90.39 |
| optimization | Group 2 | | | | | |
| (negative | | | | | | |
| control | | | | | | |
| group) | | | | | | |
| Experimental | Single- | 1 μL | 43 | 46.51 | 474.57 | 93.25 |
| group after | stranded | | | | | |
| WGBS | circular | | | | | |
| optimization | library 1 | | | | | |
| | Single- | 1 μL | | | 459.67 | 92.37 |
| | stranded | | | | | |
| | circular | | | | | |
| | library 2 | | | | | |
| WGS library | PCR Free | 0 μL | 42 | 0 | 459.03 | 93.41 |
| control | library 1 | | | | | |
| group | PCR Free | 0 μL | | | 422.95 | 94.68 |
| (positive | library 2 | | | | | |
| control | | | | | | |
| group) | | | | | | |

Example 8 indicates that when the working concentration of Tth SSB further added to the DNB preparation reaction is 46.51 ng/μL, the use of MGISEQ-2000 sequencing optimized with the base imbalance sequencing algorithm and the corresponding sequencing script can significantly improve the sequencing quality and the sequencing data yield of the whole-genome methylation single-stranded circular library.

### Example 9: Optimization Experiment for Improving Circularization Efficiency and Sequencing Quality of Whole-Genome Methylation Single-Stranded Circular Library

The reagents used in this experiment are shown in Table 17.

TABLE 17
Reagents Required for DNB Preparation and Sequencing of
Whole-Genome Methylation Single-Stranded Circular Library

| Reagent name | Brand | Cat. No. |
|---|---|---|
| DNBSEQ DNB Preparation Kit | MGI | 1000016115 |
| (included in the subsequent | | |
| sequencing kit set) | | |
| Tth SSB (500 ng/uL) | BGI | 1000007885 |
| Qubit ® ssDNA Assay Kit | Invitrogen | Q10212 |
| MGISEQ-2000RS High-throughput | MGI | 1000012554 |
| Sequencing Set (PE100) | | |

The specific experimental procedure was as follows:
1) 13 ng of whole-genome methylation single-stranded circular library was added to a new 0.2 mL PCR tube. As shown in Table 18, the single-stranded circular libraries for the control group and Optimized Scheme 2 were from the same tube, with no further additives added during single-stranded circularization; while those for Optimized Scheme 1 and Optimized Scheme 3 were from another same tube, with 1.5 μL of SSB (500 ng/μL) further added during single-stranded circularization. The volume was made up to 10 μL with Low TE Buffer, and 10 μL of DNB preparation buffer was added.
2) After mixing and centrifugation, denaturation and annealing reactions were performed using a PCR instrument at 95° C. for 1 min, 65° C. for 1 min, 40° C. for 1 min, and then holding at 4° C.
3) After the reaction was completed, the reaction solution was briefly centrifuged, and the following reagents were sequentially added to the PCR tube from the previous step on ice: 20 μL of DNB polymerase mixture I, 2 μL of DNB polymerase mixture II (LC), followed by the addition of 1 μL of additive SSB (500 ng/μL) if needed.
4) After mixing and centrifugation, the DNB preparation reaction was performed using a PCR instrument at 30° C. for 15 min and then holding at 4° C.
5) After the temperature of the PCR instrument reached 4° C., 10 μL of DNB stop buffer was immediately added. The mixture was gently mixed by pipetting 5-8 times with wide-bore pipette tips, with no shaking or vigorous pipetting allowed, and stored at 4° C. for later use.
6) The single-stranded circular DNA was quantified using the Qubit® ssDNA Assay Kit (a fluorescent quantification kit).
7) Then, 450 ng/lane of DNB was loaded onto the chip and sequenced using the optimized version of MGISEQ-2000 PE100 (with optimized sequencing algorithm and sequencing script).
8) The sequencing results are shown in Table 18. It can be seen that the sequencing quality, Total Reads yield, and Total Q30% of the methylation library with 1 μL of SSB (500 ng/uL) added during DNB preparation (Optimized Scheme 2) were significantly improved compared with the control group without addition. Meanwhile, compared with the control group, both the circularization efficiency and sequencing quality of Optimized Scheme 3 (with 1.5 ul of SSB (500 ng/uL) added during circularization and 1 μL of SSB (500 ng/uL) added during DNB preparation) were significantly improved.

TABLE 18
DNB Preparation Optimized Sequencing Results of Whole-Genome Methylation Single-Stranded Circular Library
Using Optimized Version Of MGISEQ-2000 PE100 (With Optimized Sequencing Algorithm and Sequencing Script)

| | | Volume | | | Volume | | | | |
|---|---|---|---|---|---|---|---|---|---|
| | | of Tth | | Working | of Tth | | | | |
| | | SSB (500 | | concentration | SSB | Working | | | |
| | | ng/uL) | | of Tth SSB | (500 | concentration | | | |
| | | further | | further | ng/uL) | of Tth SSB | | | |
| | | added to | | added to | further | further | | | |
| | | single- | | single- | added | added to | | | |
| | | stranded | | stranded | to DNB | DNB | | Total | Total |
| Experimental | Library | circularization | | circularization | preparation | preparation | Circularization | Reads | Q30 |
| design | name | reaction | | reaction | reaction | reaction | efficiency | (M) | (%) |
| Control | Control | 0 | μL | 0 | 0 μL | 0 | 17.8% | 388.6 | 89.51 |
| group | group | | | ng/uL | | ng/uL | | | |
| before | | | | | | | | | |
| WGBS | | | | | | | | | |
| optimization | | | | | | | | | |
| (negative | | | | | | | | | |
| control | | | | | | | | | |
| group) | | | | | | | | | |
| Experimental | Optimized | 1.5 | μL | 12.18 | 0 μL | 0 | 26.6% | 383.72 | 89.49 |
| Group- | Scheme 1 | | | ng/uL | | ng/uL | | | |
| Scheme 1 | | | | | | | | | |
| after | | | | | | | | | |
| WGBS | | | | | | | | | |
| optimization | | | | | | | | | |
| Experimental | Optimized | 0 | μL | 0 | 1 μL | 11.63 | 17.8% | 412.26 | 90.37 |
| Group- | Scheme 2 | | | ng/uL | | ng/uL | | | |
| Scheme 2 | | | | | | | | | |
| after | | | | | | | | | |
| WGBS | | | | | | | | | |
| optimization | | | | | | | | | |
| Experimental | Optimized | 1.5 | μL | 12.18 | 1 μL | 11.63 | 26.6% | 408.44 | 90.19 |
| Group- | Scheme 3 | | | ng/uL | | ng/uL | | | |
| Scheme 3 | | | | | | | | | |
| after | | | | | | | | | |
| WGBS | | | | | | | | | |
| optimization | | | | | | | | | |

Example 9 indicates that when the working concentration of Tth SSB further added in the single-stranded circularization reaction is 12.18 ng/uL, the circularization efficiency of the whole-genome methylation single-stranded circular library can be significantly improved; when the working concentration of Tth SSB further added to the DNB preparation reaction is 11.63 ng/uL, the use of MGISEQ-2000 sequencing optimized with the base imbalance sequencing algorithm and the corresponding sequencing script can improve the sequencing quality and the sequencing data yield of the whole-genome methylation single-stranded circular library. The addition of Tth SSB in both the single-stranded circularization reaction and the DNB preparation reaction can improve the circularization efficiency of the whole-genome methylation single-stranded circular library, as well as its sequencing quality and the sequencing data yield.

### Example 10: Optimization Experiment for Improving App-A Conversion Circularization Efficiency and Sequencing Quality of Third-Party Whole-Genome Methylation Library (Constructed Based on Illumina Adapters)

The reagents used in this experiment are shown in Table 19.

TABLE 19
Reagents required for App-A conversion, single-stranded
circular library preparation, and sequencing of
third-party whole-genome methylation library

| Reagent name | Brand | Cat. No. |
|---|---|---|
| MGIEasy APP-A Circularization | MGI | 1000005662 |
| Module | | |
| MGIEasy DNA Purification | MGI | 1000005279 |
| Magnetic Bead Kit | | |
| Tth SSB (500 ng/uL) | BGI | 1000007885 |
| Qubit ® ssDNA Assay Kit | Invitrogen | Q10212 |
| MGISEQ-2000RS High-throughput | MGI | 1000012555 |
| Sequencing Set (PE150) | | |

The specific experimental procedure was as follows:
1) 231 ng of the third-party whole-genome methylation App-A conversion PCR product from the library constructed based on Illumina adapters (two PCR protocols: the first protocol was 6 PCR cycles, and the second protocol was 8 PCR cycles) was transferred into a new 0.2 mL PCR tube, and the total volume was made up to 48 μL with TE Buffer.
2) After mixing and centrifugation, the PCR tube from the previous step was placed in a PCR instrument for reaction at 95° C. for 3 min and then 4° C. for 5 min. After the reaction was completed, the PCR tube was immediately transferred onto ice and briefly centrifuged.
3) A single-stranded circularization reaction solution was prepared as follows: 11.6 μL/tube of Splint Buffer (a circularization buffer) and 0.5 μL/tube of DNA Rapid Ligase were added to the reaction solution obtained in the previous step. As shown in Table 20, no additives were added to the control group, while 1.5 μL of SSB was further added to the optimization group. After mixing and centrifugation, the PCR tube was placed in a PCR instrument to perform the single-stranded circularization reaction at 37° C. for 30 min and then holding at 4° C.
4) After the reaction was completed, the reaction solution was briefly centrifuged, and the PCR tube was transferred onto ice to proceed to the next step of enzymatic digestion.
5) 1.4 μL/tube of Digestion Buffer and 2.6 μL/tube of Digestion Enzyme were added to the circularized product. After mixing and centrifugation, the PCR tube was placed in a PCR instrument to perform the enzymatic digestion reaction at 37° C. for 30 min and then holding at 4° C.
6) After the enzymatic digestion reaction was completed, the reaction solution was briefly centrifuged, and the PCR tube was transferred onto ice. Then, 7.5 μL of Digestion Stop Buffer was added to the PCR tube, vortexed three times for 3 s each time, and briefly centrifuged to collect the reaction solution at the bottom of the tube. The entire reaction solution was transferred into a new 1.5 mL centrifuge tube.
7) The reaction solution was purified using 170 μL of MGIEasy DNA Purification Magnetic Beads and eluted in 22 μL of TE Buffer. Then, 20 μL of the supernatant was transferred into a new 1.5 mL centrifuge tube.
8) The single-stranded circular DNA was quantified using the Qubit® ssDNA Assay Kit (a fluorescent quantification kit), and the quantification results are shown in Table 20. Compared with the circularization control group with no SSB added, the circularization efficiency of the single-stranded circular library in the circularization optimization group with SSB added was significantly improved.

TABLE 20
Single-Stranded Circularization Results of Third-Party Whole-Genome Methylation
Library Conversion PCR Product Before and After Optimization

| | | Volume | | | | | Total | |
|---|---|---|---|---|---|---|---|---|
| | | of Tth | Working | PCR | Total | Single- | single- | |
| | | SSB | concentration | library | input | stranded | stranded | |
| | | (500 | of Tth | main | amount | circular | circular | Single- |
| | | ng/uL) | SSB | peak | for | library | library | stranded |
| Experimental | Library | further | further | size | circularization | concentration | amount | circularization |
| design | name | added | added | (bp) | (ng) | (ng/μL) | (ng) | efficiency |
| PCR 6 | Control | 0 | 0 | 300 | 231 | 2.41 | 53.02 | 23% |
| cycles- | Group | | ng/uL | | | | | |
| circularization | 1-1 | | | | | | | |
| control | | | | | | | | |
| group | | | | | | | | |
| PCR 6 | SSB- | 1.5 μL | 12.18 | 300 | 231 | 3.07 | 67.54 | 29.2% |
| cycles- | 1.5 μL | | ng/uL | | | | | |
| circularization | 1-1 | | | | | | | |
| optimization | | | | | | | | |
| group | | | | | | | | |
| PCR 8 | Control | 0 | 0 | 300 | 231 | 3.29 | 72.38 | 31.3% |
| cycles- | Group | | ng/uL | | | | | |
| circularization | 2-1 | | | | | | | |
| control | | | | | | | | |
| group | | | | | | | | |
| PCR 8 | SSB- | 1.5 μL | 12.18 | 300 | 231 | 3.7 | 81.4 | 35.2% |
| cycles- | 1.5 μL | | ng/uL | | | | | |
| circularization | 2-1 | | | | | | | |
| optimization | | | | | | | | |
| group | | | | | | | | |

9) DNB preparation and sequencing: 12 ng of the constructed single-stranded circular DNA library was used for DNB preparation. 450 ng/lane of DNB was loaded onto the chip and sequenced using the optimized version of MGISEQ-2000 PE150 (with optimized sequencing algorithm and sequencing script).
10) The sequencing results are shown in Table 21. The Total Reads yield and Total Q30% of the third-party methylation library in the optimization group with 1.5 μL of SSB (500 ng/uL) further added to the single-stranded circularization step were significantly improved compared with the control group without addition.

TABLE 21
Sequencing Results of Third-Party Whole-Genome Methylation Single-
Stranded Circular Library Before and After Circularization Optimization
Using Optimized Version Of MGISEQ-2000 PE150 (With Optimized
Sequencing Algorithm and Sequencing Script)

| | | | Working | | |
|---|---|---|---|---|---|
| | | Volume of Tth | concentration of Tth | | |
| | | SSB further added | SSB further added to | | |
| | | to single-stranded | single-stranded | Total | Total |
| Experimental | Library | circularization | circularization | Reads | Q30 |
| design | name | reaction | reaction | (M) | (%) |
| PCR 6 cycles- | Control | 0 μL | 0 ng/μL | 310.75 | 86.76 |
| circularization | Group | | | | |
| control group | 1-1 | | | | |
| PCR 6 cycles- | SSB- | 1.5 μL | 12.18 ng/μL | 407.79 | 91.45 |
| circularization | 1.5 μL | | | | |
| optimization | 1-1 | | | | |
| group | | | | | |
| PCR 8 cycles- | Control | 0 μL | 0 ng/μL | 331.14 | 88.07 |
| circularization | Group | | | | |
| control group | 2-1 | | | | |
| PCR 8 cycles- | SSB- | 1.5 μL | 12.18 ng/μL | 402.65 | 91.24 |
| circularization | 1.5 μL | | | | |
| optimization | 2-1 | | | | |
| group | | | | | |

Example 10 indicates that when the working concentration of Tth SSB further added in the single-stranded circularization reaction is 12.18 ng/uL, the circularization efficiency of the whole-genome methylation single-stranded circular library converted based on the Illumina library construction method, as well as the sequencing quality and the sequencing data yield, can be significantly improved.

### Example 11: Optimization Experiment for Improving Sequencing Quality of Whole-Genome Methylation Single-Stranded Circular Library

The reagents used in this experiment are shown in Table 22.

TABLE 22
Reagents required for DNB preparation and sequencing of
whole-genome methylation single-stranded circular library

| Reagent name | Brand | Cat. No. |
|---|---|---|
| DNBSEQ DNB Preparation Kit | MGI | 1000016115 |
| (included in the subsequent | | |
| sequencing kit set) | | |
| DMSO (dimethyl sulfoxide) | Sigma | D2650 |
| Formamide | Diamond | A100314-0100 |
| Betaine | BBI | A600185 |
| Qubit ® ssDNA Assay Kit | Invitrogen | Q10212 |
| MGISEQ-2000RS High-throughput | MGI | 1000012554 |
| Sequencing Set (PE100) | | |

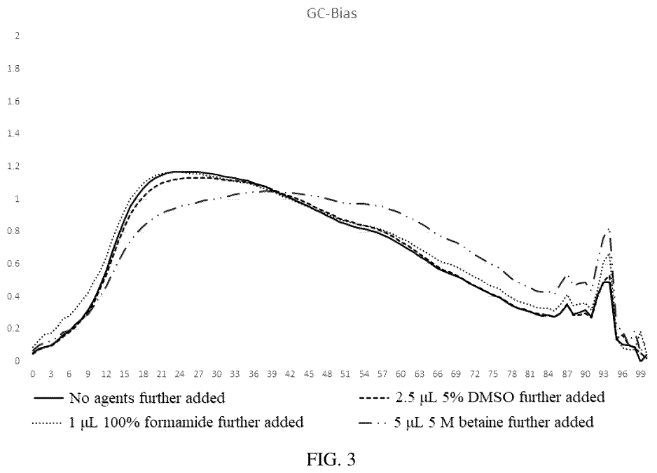

The specific experimental procedure was as follows:
1) 8 ng of whole-genome methylation single-stranded circular library was added to a new 0.2 mL PCR tube. Then, for Experimental Group Schemes 1, 2, and 3, 2.5 μL of 5% DMSO, 1 μL of 100% formamide, and 5 μL of 5M betaine were further added as additives, respectively, as shown in Table 23, and the volume was made up to 10 μL with Low TE Buffer, followed by the addition of 10 μL of DNB preparation buffer.
2) After mixing and centrifugation, denaturation and annealing reactions were performed using a PCR instrument at 95° C. for 1 min, 65° C. for 1 min, 40° C. for 1 min, and then holding at 4° C.
3) After the reaction was completed, the reaction solution was briefly centrifuged, and the following reagents were sequentially added to the PCR tube from the previous step on ice: 20 μL of DNB polymerase mixture I and 2 μL of DNB polymerase mixture II (LC).
4) After mixing and centrifugation, the DNB preparation reaction was performed using a PCR instrument at 30° C. for 15 min and then holding at 4° C.
5) After the temperature of the PCR instrument reached 4° C., 10 μL of DNB stop buffer was immediately added. The mixture was gently mixed by pipetting 5-8 times with wide-bore pipette tips, with no shaking or vigorous pipetting allowed, and stored at 4° C. for later use.
6) DNB quantification was performed using the Qubit® ssDNA Assay Kit (a fluorescent quantification kit).
7) Then, all of DNB was loaded onto the chip and sequenced using the optimized version of MGISEQ-2000 PE100 (with optimized sequencing algorithm and sequencing script).
8) The sequencing results are shown in Table 23. It can be seen that compared with the control group with no additives added during DNB preparation, the experimental groups with three additives (DMSO, formamide, and betaine) added did not exhibit a significant improvement in the sequencing quality and Total Reads yield of the methylation library.
9) Meanwhile, as shown in FIG. 3, compared with the control group, the sequencing quality (GC-Bias) of Experimental Group Scheme 3 (with 5 μL of 5M betaine added during DNB preparation) was significantly improved.

TABLE 23
DNB Preparation Optimized Sequencing Results of Whole-Genome Methylation
Single-Stranded Circular Library Using Optimized Version Of MGISEQ-2000
PE100 (With Optimized Sequencing Algorithm and Sequencing Script)

| | | Further additive | Working | | |
|---|---|---|---|---|---|
| | | and volume | concentration | Total | Total |
| Experimental | Library | for DNB | of additive | Reads | Q30 |
| design | name | preparation | further added | (M) | (%) |
| Control group | Control | \ | 0 | 347.5 | 89.39 |
| before WGBS | group | | | | |
| optimization | | | | | |
| Experimental | Optimized | 5% DMSO | 0.30% | 361.03 | 89.91 |
| Group-Scheme | Scheme 1 | 2.5 μL | DMSO | | |
| 1 after WGBS | | | | | |
| optimization | | | | | |
| Experimental | Optimized | 100% | 2.38% | 323.15 | 89.91 |
| Group-Scheme | Scheme 2 | formamide | formamide | | |
| 2 after WGBS | | 1 μL | | | |
| optimization | | | | | |
| Experimental | Optimized | 5M betaine | 0.60M | 341.13 | 89.87 |
| Group-Scheme | Scheme 3 | 5 μL | betaine | | |
| 3 after WGBS | | | | | |
| optimization | | | | | |

Example 11 indicates that the further addition of DMSO, formamide, and betaine in the DNB preparation reaction cannot significantly improve the sequencing quality of the whole-genome methylation single-stranded circular library; however, when the working concentration of betaine is 0.60 M, the coverage uniformity of high-GC regions in the whole-genome methylation single-stranded circular library can be significantly improved.

### Example 12: Optimization Experiment for Improving Sequencing Quality of Whole-Genome Methylation Single-Stranded Circular Library

The reagents used in this experiment are shown in Table 24.

TABLE 24
Reagents Required for DNB Preparation and Sequencing of
Whole-Genome Methylation Single-Stranded Circular Library

| Reagent name | Brand | Cat. No. |
|---|---|---|
| DNBSEQ DNB Preparation Kit | MGI | 1000016115 |
| (included in the subsequent | | |
| sequencing kit set) | | |
| Tth SSB (2 ug/uL) | BGI | 1000007886 |
| Betaine | BBI | A600185 |
| Qubit ® ssDNA Assay Kit | Invitrogen | Q10212 |
| MGISEQ-2000RS High-throughput | MGI | 1000012555 |
| Sequencing Set (PE150) | | |

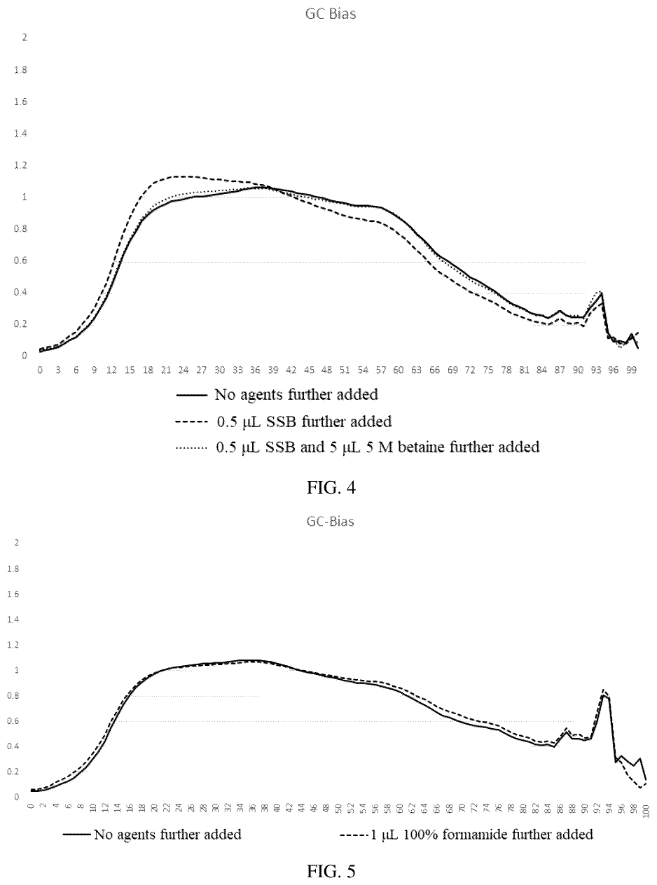

The specific experimental procedure was as follows:
1) 12 ng of whole-genome methylation single-stranded circular library was added to a new 0.2 mL PCR tube. As shown in Table 25, for Experimental Group Scheme 2, 5 μL of 5M betaine was further added, and the volume was made up to 10 μL with Low TE Buffer for each reaction, followed by the addition of 10 μL of DNB preparation buffer.
2) After mixing and centrifugation, denaturation and annealing reactions were performed using a PCR instrument at 95° C. for 1 min, 65° C. for 1 min, 40° C. for 1 min, and then holding at 4° C.
3) After the reaction was completed, the reaction solution was briefly centrifuged, and the following reagents were added to the PCR tube of the control group from the previous step on ice: 20 μL of DNB polymerase mixture I and 2 μL of DNB polymerase mixture II (LC). Further, the following reagents were added to the PCR tubes of the two experimental groups from the previous step: 1 μL of Tth SSB (2 ug/uL), 19.5 μL of DNB polymerase mixture I, and 2 μL of DNB polymerase mixture II (LC).
4) After mixing and centrifugation, the DNB preparation reaction was performed using a PCR instrument at 30° C. for 15 min and then holding at 4° C.
5) After the temperature of the PCR instrument reached 4° C., 10 μL of DNB stop buffer was immediately added. The mixture was gently mixed by pipetting 5-8 times with wide-bore pipette tips, with no shaking or vigorous pipetting allowed, and stored at 4° C. for later use.
6) DNB quantification was performed using the Qubit® ssDNA Assay Kit (a fluorescent quantification kit).
7) Then, half the volume of DNB was loaded onto the chip and sequenced using the optimized version of MGISEQ-2000 PE150 (with optimized sequencing algorithm and sequencing script).
8) As shown in Table 25 and FIG. 4, compared with the control group, the addition of SSB (2 ug/uL) during DNB preparation (Optimized Scheme 1) significantly increased Total reads and Q30 but decreased the high-GC region coverage uniformity; while when SSB and betaine are added in combination (Optimized Scheme 2), the effects of significantly increasing Total reads and Q30 and maintaining no decrease in high-GC region coverage compared with that of the control group can be achieved.

TABLE 25
DNB Preparation Optimized Sequencing Results of Whole-Genome Methylation
Single-Stranded Circular Library Using Optimized Version of MGISEQ-2000
PE150 (With Optimized Sequencing Algorithm and Sequencing Script)

| | | Further additive | Working | | |
|---|---|---|---|---|---|
| | | and volume | concentration | Total | Total |
| Experimental | Library | for DNB | of additive | Reads | Q30 |
| design | name | preparation | further added | (M) | (%) |
| Control group | Control | \ | 0 | 365.85 | 87.99 |
| before WGBS | group | | | | |
| optimization | | | | | |
| Experimental | Optimized | Tth SSB (2 | 46.51 ng/uL | 433.65 | 89.3 |
| Group-Scheme | Scheme 1 | ug/uL) 1 μL | Tth SSB | | |
| 1 after WGBS | | | | | |
| optimization | | | | | |
| Experimental | Optimized | Tth SSB (2 | 46.51 ng/uL | 426.98 | 89.89 |
| Group-Scheme | Scheme 2 | ug/uL) 1 μL + | Tth SSB + | | |
| 2 after WGBS | | 5M betaine | 0.58M betaine | | |
| optimization | | 5 μL | | | |

Example 12 indicates that the further addition of the combined formulation of Tth SSB (working concentration 46.51 ng/uL) and betaine (working concentration 0.58 M) in the DNB preparation reaction can significantly improve the sequencing quality and the sequencing data yield of the whole-genome methylation single-stranded circular library, as well as the coverage uniformity of high-GC regions in the whole-genome methylation single-stranded circular library.

### Example 13: Optimization Experiment for Improving Sequencing Quality of Whole-Genome Methylation Double-Stranded Circular Library

The reagents used in this experiment are shown in Table 26.

TABLE 26
Reagents Required for DNB Preparation and Sequencing of
Whole-Genome Methylation Double-Stranded Circular Library

| Reagent name | Brand | Cat. No. |
|---|---|---|
| DNBSEQ DNB Preparation Kit | MGI | 1000016115 |
| (included in the subsequent | | |
| sequencing kit set) | | |
| WGBS DNB Buffer | MGI | 940-000308-00 |
| Formamide | Diamond | A100314-0100 |
| Qubit ® ssDNA Assay Kit | Invitrogen | Q10212 |
| MGISEQ-2000RS High-throughput | MGI | 1000012555 |
| Sequencing Set (PE150) | | |

The specific experimental procedure was as follows:
1) 23.76 ng of whole-genome methylation double-stranded circular library was added to a new 0.2 mL PCR tube. As shown in Table 27, for the experimental group, 1 μL of 100% formamide was further added, and the volume was made up to 10 μL with Low TE Buffer for each reaction, followed by the addition of 10 μL of WGBS DNB Buffer.
2) After mixing and centrifugation, the following reagents were sequentially added to the PCR tube from the previous step on ice: 20 μL of DNB polymerase mixture I and 2 μL of DNB polymerase mixture II (LC).
3) After mixing and centrifugation, the DNB preparation reaction was performed using a PCR instrument at 30° C. for 15 min and then holding at 4° C.
4) After the temperature of the PCR instrument reached 4° C., 10 μL of DNB stop buffer was immediately added. The mixture was gently mixed by pipetting 5-8 times with wide-bore pipette tips, with no shaking or vigorous pipetting allowed, and stored at 4° C. for later use.
5) DNB quantification was performed using the Qubit® ssDNA Assay Kit (a fluorescent quantification kit).
6) Then, 25 μL of DNB was loaded onto the chip and sequenced using the optimized version of MGISEQ-2000 PE150 (with optimized sequencing algorithm and sequencing script).
7) The sequencing results are shown in Table 27 and FIG. 5. The sequencing quality and Total Reads yield of the methylation library in the experimental group with 1 μL of 100% formamide added during DNB preparation were significantly improved compared with the control group without addition, and the high-GC coverage was slightly improved.

TABLE 27
DNB Preparation Optimized Sequencing Results of Whole-Genome Methylation
Double-Stranded Circular Library Using Optimized Version Of MGISEQ-2000
PE150 (With Optimized Sequencing Algorithm and Sequencing Script)

| | | Further additive | Working | | |
|---|---|---|---|---|---|
| | | and volume | concentration | Total | Total |
| Experimental | Library | for DNB | of additive | Reads | Q30 |
| design | name | preparation | further added | (M) | (%) |
| Control group | Control | \ | 0 | 348.67 | 85.07 |
| before WGBS | group | | | | |
| optimization | | | | | |
| Experimental | Optimized | 100% | 2.38% | 431.84 | 89.19 |
| group after | Scheme | formamide | formamide | | |
| WGBS | | 1 μL | | | |
| optimization | | | | | |

Example 13 indicates that the further addition of formamide (working concentration 2.38%) in the DNB preparation reaction can not only significantly improve the sequencing quality and the sequencing data yield of the whole-genome methylation double-stranded circular library, but also slightly improve the coverage uniformity of high-GC regions in the whole-genome methylation double-stranded circular library.

### Example 14: Optimization Experiment for Improving Single-Stranded Circularization Efficiency and Sequencing Quality of Whole-Genome PCR-Free Library

The reagents used in this experiment are shown in Table 28.

TABLE 28
Reagents Required for Single-Stranded Circularization
Experiment of Whole-Genome PCR-Free Library

| Reagent name | Brand | Cat. No. |
|---|---|---|
| MGIEasy Circularization | MGI | 1000005260 |
| Module | | |
| MGIEasy DNA Purification | MGI | 1000005279 |
| Magnetic Bead Kit | | |
| Tth SSB (500 ng/uL) | BGI | 1000007885 |
| Tth SSB (2 ug/uL) | BGI | 1000007886 |
| Qubit ® ssDNA Assay Kit | Invitrogen | Q10212 |
| MGISEQ-2000RS High-throughput | MGI | 1000012555 |
| Sequencing Set (PE150) | | |

The specific experimental procedure was as follows:
1) 167 ng of whole-genome PCR-free ligation product (after fragmentation and single selection) was transferred into a new 0.2 mL PCR tube, and the total volume was made up to 48 μL with TE Buffer.
2) After mixing and centrifugation, the PCR tube from the previous step was placed in a PCR instrument for reaction at 95° C. for 3 min and then 4° C. for 10 min. After the reaction was completed, the PCR tube was immediately transferred onto ice and briefly centrifuged.
3) A single-stranded circularization reaction solution was prepared as follows: 11.5 μL/tube of Splint Buffer (a circularization buffer) and 0.5 μL/tube of DNA Rapid Ligase were added to the reaction solution obtained in the previous step. As shown in Table 29, 1 μL/tube of Tth SSB (500 ng/uL) was further added to the experimental group. After mixing and centrifugation, the PCR tube was placed in a PCR instrument to perform the single-stranded circularization reaction at 37° C. for 10 min and then holding at 4° C.
4) After the reaction was completed, the reaction solution was briefly centrifuged, and the PCR tube was transferred onto ice to proceed to the next step of enzymatic digestion.
5) 1.4 μL/tube of Digestion Buffer and 2.6 μL/tube of Digestion Enzyme were added to the circularized product. After mixing and centrifugation, the PCR tube was placed in a PCR instrument to perform the enzymatic digestion reaction at 37° C. for 10 min and then holding at 4° C.
6) After the enzymatic digestion reaction was completed, the reaction solution was briefly centrifuged, and the PCR tube was transferred onto ice. Then, 7.5 μL of Digestion Stop Buffer was added to the PCR tube, vortexed three times for 3 s each time, and briefly centrifuged to collect the reaction solution at the bottom of the tube. The entire reaction solution was transferred into a new 1.5 mL centrifuge tube.
7) The reaction solution was purified using 130 μL of MGIEasy DNA Purification Magnetic Beads and eluted in 25 μL of TE Buffer.
8) The single-stranded circular DNA was quantified using the Qubit® ssDNA Assay Kit (a fluorescent quantification kit), and the quantification results are shown in Table 29. Compared with the control group with no SSB added, the circularization efficiency of the whole-genome PCR-free single-stranded circular library in the experimental group with 1 μL of Tth SSB (500 ng/μL) further added was significantly improved.

TABLE 29
Single-Stranded Circularization Results of Whole-Genome PCR-Free Library Before and After Optimization

| | | Working | | | | | |
|---|---|---|---|---|---|---|---|
| | | concentration | | | | | |
| | | of Tth SSB | | | | | |
| | | (500 | | | | | |
| | | ng/uL) | PCR- | | | Total | |
| | | further | free | Total | Single- | single- | |
| | Additive | added to | library | input | stranded | stranded | |
| | in single- | single- | main | amount | circular | circular | Single- |
| | stranded | stranded | peak | for | library | library | stranded |
| Library | circularization | circularization | size | circularization | concentration | amount | circularization |
| name | reaction | reaction | (bp) | (ng) | (ng/μL) | (ng) | efficiency |
| Control | None | 0 | 650 | 167 | 1.81 | 45.25 | 27.2% |
| Group-1 | | | | | | | |
| Control | | | 650 | 167 | 1.78 | 44.5 | 26.7% |
| Group-2 | | | | | | | |
| Experimental | 1 μL of | 8.20 | 650 | 167 | 2.07 | 51.75 | 31.1% |
| Group--1 | Tth SSB | ng/uL | | | | | |
| Experimental | (500 | Tth SSB | 650 | 167 | 2.13 | 53.25 | 32% |
| Group--2 | ng/uL) | | | | | | |
| | further | | | | | | |
| | added | | | | | | |

9) DNB preparation was performed using the DNB preparation reagents included in the MGISEQ-2000RS High-throughput Sequencing Set (PE150). 5 ng of single-stranded circular library was taken, and the volume was made up to 10 μL with TE Buffer, followed by the addition of 10 μL/tube of DNB preparation buffer.
12) After mixing and centrifugation, denaturation and annealing reactions were performed using a PCR instrument at 95° C. for 1 min, 65° C. for 1 min, 40° C. for 1 min, and then holding at 4° C.
13) After the reaction was completed, the reaction solution was briefly centrifuged, and the following reagents were sequentially added to the PCR tube from the previous step on ice: 20 μL of DNB polymerase mixture I and 2 μL of DNB polymerase mixture II (LC). As shown in Table 30, no Tth SSB was added to the control group, while 1 μL/tube of Tth SSB (2 ug/uL) was added to the experimental group.
14) After mixing and centrifugation, the DNB preparation reaction was performed using a PCR instrument at 30° C. for 25 min and then holding at 4° C.
15) When the temperature of the PCR instrument reached 4° C., 10 μL of DNB stop buffer was immediately added. The mixture was gently mixed by pipetting 5-8 times with wide-bore pipette tips, with no shaking or vigorous pipetting allowed, and stored at 4° C. for later use.
16) DNB quantification was performed using the Qubit® ssDNA Assay Kit (a fluorescent quantification kit).
17) 400 ng of DNB was loaded onto the chip and sequenced using the optimized version of MGISEQ-2000 PE150 (with optimized sequencing algorithm and sequencing script).
18) The DNB quantification and sequencing results are shown in Table 31. Compared with the control group without addition, the PCR-free library with 1 μL of SSB (2 ug/μL) added in both the single-stranded circularization step and DNB preparation step exhibited a certain improvement in Total Reads yield and Total Q30%;
19) 95G of sequencing data was extracted and analyzed using the MGI WGAA2.1 WGS analysis software (reference genome version hg19). The analysis results are shown in Table 31. It can be seen that the coverage uniformity and variant detection performance of the experimental group (1 μL added in both circularization and DNB preparation steps) were improved, as reflected in the increase in 20× coverage value at 30× depth and the improvement in SNP and InDel F-measure.

TABLE 30
Sequencing Results of Whole-Genome PCR-Free Library Before and After Optimization
On MGISEQ-2000 PE150 (With Optimized Sequencing Algorithm and Sequencing Script)

| | | | Volume of | | | |
|---|---|---|---|---|---|---|
| | | | SSB (2 | | | |
| | Volume of | | ug/uL) | | | |
| | SSB (500 | Working | further | Working | | |
| | ng/uL) further | concentration | added to | concentration | | |
| | added to | of Tth SSB | DNB | of Tth SSB | Total | Total |
| Experimental | circularization | (500 ng/uL) | preparation | (2 ug/uL) | Reads | Q30 |
| design | step | further added | step | further added | (M) | (%) |
| Control | 0 μL | 0 | 0 μL | 0 | 380.84 | 88.31 |
| Group-1 | | | | | | |
| Control | | | | | 382.67 | 88.57 |
| Group-2 | | | | | | |
| Experimental | 1 μL | 8.20 ng/uL | 1 μL | 46.51 ng/uL | 401.27 | 92.96 |
| Group- | | Tth SSB | | Tth SSB | | |
| Scheme 1-1 | | | | | | |
| Experimental | | | | | 402.65 | 93.24 |
| Group- | | | | | | |
| Scheme 1-2 | | | | | | |

TABLE 31
WGS Data Analysis Results of Whole-Genome PCR-Free Library
Before and After Optimization On MGISEQ-2000 PE150 (With
Optimized Sequencing Algorithm and Sequencing Script)

| | Clean | Mapping | Average | | 20x | | InDel |
|---|---|---|---|---|---|---|---|
| Experimental | Q30 | rate | depth | Coverage | Coverage | SNP F- | F- |
| design | (%) | (%) | (X) | (%) | (%) | measure | measure |
| Control Group-1 | 88.30 | 99.57 | 31.57 | 99.01 | 91.07 | 0.9956 | 0.9797 |
| Control Group-2 | 89.71 | 99.58 | 31.76 | 99.02 | 91.23 | 0.9957 | 0.9798 |
| Experimental | 91.52 | 99.60 | 32.01 | 99.04 | 93.37 | 0.9964 | 0.9869 |
| Group-Scheme | | | | | | | |
| 1-1 | | | | | | | |
| Experimental | 91.73 | 99.61 | 32.06 | 99.04 | 93.40 | 0.9964 | 0.9870 |
| Group-Scheme | | | | | | | |
| 1-2 | | | | | | | |

Example 14 indicates that the further addition of Tth SSB (working concentrations 8.20 ng/uL and 46.51 ng/uL, respectively) in the circularization reaction and DNB preparation reaction can not only significantly improve the circularization efficiency of the whole-genome PCR-free library, but also slightly improve the sequencing quality and the sequencing data yield, as well as improve the coverage and variant detection effect of the whole-genome PCR-free library.

In the description of this specification, reference to the terms “an embodiment”, “some embodiments”, “an example”, “a specific example”, or “some examples” and the like means that the specific features, structures, materials, or characteristics described in combination with the embodiment(s) or example(s) are included in at least one embodiment or example of the present disclosure. In this specification, the schematic representations of the above terms are not necessarily directed to the same embodiment(s) or example(s). Furthermore, the described specific features, structures, materials, or characteristics may be combined in a suitable manner in any one or more embodiments or examples. In addition, without mutual contradiction, those skilled in the art may incorporate and combine different embodiments or examples and features of the different embodiments or examples described in this specification.

Although the embodiments of the present disclosure have been shown and described above, it can be understood that the above embodiments are exemplary and should not be construed as limitations on the present disclosure, and those of ordinary skill in the art can make changes, modifications, substitutions and variations to the above embodiments within the scope of the present disclosure.

What is claimed is:

1. A method for preparing DNA nanoballs (DNB) or single-stranded linear DNA, the method comprising:
(a) performing a circularization reaction on a linear DNA library to obtain a circular DNA library, wherein the circularization reaction comprises a single-stranded circularization reaction or a double-stranded circularization reaction, and wherein the circular DNA library comprises a single-stranded circular DNA library or a double-stranded circular DNA library; and
(b) performing a rolling circle amplification reaction on the circular DNA library to obtain DNB,
wherein when the circularization reaction in step (a) is a single-stranded circularization reaction,
step (a) further comprises: adding at least one of the following additives in the single-stranded circularization reaction to obtain a single-stranded circular DNA library:
1. an additive capable of reducing DNA secondary structure; or
2. an additive capable of reducing DNA secondary structure and maintaining stability of single-stranded DNA, and/or
step (b) further comprises: performing a rolling circle amplification reaction using the single-stranded circular DNA library from step (a), wherein at least one of the following additives is added in the rolling circle amplification reaction:
3. an additive capable of reducing DNA secondary structure; or
4) an additive capable of reducing DNA secondary structure and maintaining stability of single-stranded DNA,
wherein when the circularization reaction in step (a) is a double-stranded circularization reaction,
step (b) further comprises: performing a rolling circle amplification reaction using the double-stranded circular DNA library from step (a), wherein at least one of the following additives is added in the rolling circle amplification reaction:
5. an additive capable of reducing DNA secondary structure and maintaining stability of single-stranded DNA; or
6. an additive capable of reducing stability of double helix structure of circular double-stranded DNA.

2. The method according to claim 1, wherein the additive capable of reducing DNA secondary structure comprises at least one of a non-ionic detergent, betaine, or dimethyl sulfoxide (DMSO), optionally, the non-ionic detergent comprises at least one of Triton-X100, Tween-20, or NP40;
optionally, the additive capable of reducing DNA secondary structure and maintaining stability of single-stranded DNA comprises at least one of single-stranded DNA binding protein (SSB) and heat shock protein (HSP);
optionally, the additive capable of reducing stability of double helix structure of circular double-stranded DNA comprises at least one of formamide, ammonium sulfate, urea, or tetramethylammonium hydroxide.

3. The method according to claim 2, wherein the additive in the single-stranded circularization reaction comprises at least one of SSB, a non-ionic detergent, betaine, or DMSO;
optionally, the additive in the single-stranded circularization reaction is a mixture of a non-ionic detergent and betaine;
optionally, the additive in the single-stranded circularization reaction is a mixture of a non-ionic detergent and DMSO;
optionally, the additive in the single-stranded circularization reaction is a mixture of SSB and a non-ionic detergent and/or betaine.

4. The method according to claim 1, wherein the additive in the rolling circle amplification reaction for the single-stranded circular DNA library comprises at least one of SSB, or a mixture of SSB and a non-ionic detergent and/or betaine.

5. The method according to claim 2, wherein the additive in the rolling circle amplification reaction for the double-stranded circular DNA library comprises at least one or SSB or formamide.

6. The method according to claim 5, wherein a working concentration of SSB, when added in the single-stranded circularization reaction, ranges from 5 ng/μL to 50 ng/μL;
optionally, a working concentration of the non-ionic detergent, when added in the single-stranded circularization reaction, ranges from 0.05 vol % to 2.00 vol %;
optionally, a working concentration of DMSO, when added in the single-stranded circularization reaction, ranges from 1.5 vol % to 4.0 vol %;
optionally, a working concentration of betaine, when added in the single-stranded circularization reaction, ranges from 0.1 M to 1 M.

7. The method according to claim 4, wherein a working concentration of SSB, when added in the rolling circle amplification reaction for the single-stranded circular DNA library, ranges from 5 ng/μL to 130 ng/μL.

8. The method according to claim 7, wherein a working concentration of betaine, when added in the rolling circle amplification reaction for the single-stranded circular DNA library, ranges from 0.1 M to 1 M;
optionally, a working concentration of the non-ionic detergent, when added in the rolling circle amplification reaction for the single-stranded circular DNA library, ranges from 0.05 vol % to 2.00 vol %.

9. The method according to claim 5, wherein a working concentration of SSB, when added in the rolling circle amplification reaction for the double-stranded circular DNA library, ranges from 20 ng/μL to 100 ng/μL;
optionally, a working concentration of formamide, when added in the rolling circle amplification reaction for the double-stranded circular DNA library, ranges from 1.5 vol % to 4 vol %.

10. A single-stranded circularization reaction system, comprising:
oligonucleotides;
DNA ligase;
a ligation reaction buffer; and
an additive capable of reducing DNA secondary structure, and/or an additive capable of reducing DNA secondary structure and maintaining stability of single-stranded DNA.

11. The single-stranded circularization reaction system according to claim 10, wherein the DNA ligase comprises any one of T4 DNA ligase or Taq ligase.

12. The single-stranded circularization reaction system according to claim 10, further comprising a sequencing chip or magnetic beads, wherein the oligonucleotides are immobilized on the sequencing chip or on the magnetic beads and/or free in a suspension of the magnetic beads.

13. The single-stranded circularization reaction system according to claim 10, wherein the additive capable of reducing DNA secondary structure comprises at least one of a non-ionic detergent, betaine, or DMSO, optionally, the non-ionic detergent comprises at least one of Triton-X100, Tween-20, or NP40;
optionally, the additive capable of reducing DNA secondary structure and maintaining stability of single-stranded DNA comprises at least one of SSB or HSP.

14. The single-stranded circularization reaction system according to claim 10, wherein the additive in the single-stranded circularization reaction system comprises at least one of SSB, a non-ionic detergent, betaine, or DMSO;
optionally, the additive is a mixture of a non-ionic detergent and betaine;
optionally, the additive is a mixture of a non-ionic detergent and DMSO;
optionally, the additive is a mixture of SSB and a non-ionic detergent and/or betaine.

15. A rolling circle amplification reaction system for a single-stranded circular DNA library or a double-stranded circular DNA library, comprising:
a polymerase; and
a rolling circle amplification reaction buffer,
wherein in the case of the rolling circle amplification reaction system for a single-stranded circular DNA library, the system further comprises an additive capable of reducing DNA secondary structure, and/or an additive capable of reducing DNA secondary structure and maintaining stability of single-stranded DNA,
wherein in the case of the rolling circle amplification reaction system for a double-stranded circular DNA library, the system further comprises an additive capable of reducing DNA secondary structure and maintaining stability of single-stranded DNA, and/or an additive capable of reducing stability of double helix structure of circular double-stranded DNA.

16. The rolling circle amplification reaction system according to claim 15, wherein the polymerase comprises any one of Phi29 DNA polymerase or Bst DNA polymerase.

17. The rolling circle amplification reaction system according to claim 19, further comprising a sequencing chip or magnetic beads, wherein a surface of the sequencing chip is for performing the rolling circle amplification reaction, or the magnetic beads and/or a suspension of the magnetic beads are for performing a rolling circle amplification reaction.

18. The rolling circle amplification reaction system according to claim 15, wherein the additive capable of reducing DNA secondary structure comprises at least one of a non-ionic detergent, betaine, or DMSO;
optionally, the non-ionic detergent comprises at least one of Triton-X100, Tween-20, or NP40;
optionally, the additive capable of reducing DNA secondary structure and maintaining stability of single-stranded DNA comprises at least one of SSB or HSP.

19. The rolling circle amplification reaction system according to claim 15, wherein the additive capable of reducing DNA secondary structure and maintaining stability of single-stranded DNA comprises at least one of SSB, or a mixture of SSB and a non-ionic detergent and/or betaine.

20. The rolling circle amplification reaction system according to claim 15, wherein the additive capable of reducing stability of double helix structure of circular double-stranded DNA comprises at least one of formamide, ammonium sulfate, urea, or tetramethylammonium hydroxide.
